(19) United States
(12) Patent Application Publication
(10) Pub. No.: US 2015/0368599 A1
(43) Pub. Date: Dec. 24, 2015

(54) DESIGN AND HOT EMBOSSING OF MACRO AND MICRO FEATURES WITH HIGH RESOLUTION MICROSCOPY ACCESS

(71) Applicants: The Charles Stark Draper Laboratory, Inc., Cambridge, MA (US); The University of South Florida, Tampa, FL (US)

(72) Inventors: Steven Maher, Tampa, FL (US); Wajeeh M. Saadi, Tampa, FL (US); Amy Jane Taylor, St. Pete Beach, FL (US); Hoi-Cheong Steve Sun, Tampa, FL (US); Dennis Kyle, Tampa, FL (US); John Adams, Tampa, FL (US)

(21) Appl. No.: 14/743,634

(22) Filed: Jun. 18, 2015

Related U.S. Application Data
(60) Provisional application No. 62/013,854, filed on Jun. 18, 2014.
(60) Provisional application No. 62/086,012, filed on Dec. 1, 2014.
(60) Provisional application No. 62/151,292, filed on Apr. 22, 2015.

(51) Int. Cl. C12M 1/32; C08G 65/40; C12M 1/00; C09D 175/04; C09D 163/00; B29C 39/02; C08G 77/04; C08G 73/10

(52) U.S. Cl. CPC C12M 23/12; C08G 77/04; C08G 65/4012; C08G 73/10; C09D 175/04; C09D 163/00; B29C 39/026; C12M 23/22; B29K 2071/00

(57) ABSTRACT

This disclosure provides micro-feature devices and methods for fabricating micro-feature devices. A micro-feature device can include a substantially rigid transparent substrate. The device can include a plurality of macrowells defined in the transparent substrate. Each macrowell can have a width in the range of about one millimeter to about 35 millimeters and a depth in the range of about two millimeters to about 12 millimeters. Each macrowell can include a respective plurality of microwells defined in a respective lower surface of the macrowell. Each microwell can have a width in the range of about 50 microns to about 500 microns and a depth in the range of about 50 microns to about 500 microns.

### RELATED APPLICATIONS

The present application for claims priority to U.S. Provisional Application No. 62/013,854, entitled “Multiplexed Liver Model for High-Throughput Drug Screening,” filed Jun. 18, 2014; U.S. Provisional Application No. 62/086,012, entitled “Multiplexed Liver Platform for High-Throughput Screening,” filed Dec. 1, 2014; and U.S. Provisional Application No. 62/151,292, entitled “Design And Hot Embossing Of Macro And Micro Features With High Resolution Microscopy Access,” filed Apr. 22, 2015, all of which are assigned to the assignee hereof and hereby expressly incorporated by reference herein.

### BACKGROUND

Micro-feature devices can be used for performing multiplexed experiments on cell cultures. Very small features sizes for such devices can be required to successfully maintain cell cultures. However, it can be difficult to reliably form devices with features sizes that are sufficiently small. Traditional techniques for forming such devices can be time consuming and expensive.

### SUMMARY

The systems, methods, and devices of the disclosure each have several innovative aspects, no single one of which is solely responsible for the desirable attributes disclosed herein.

One innovative aspect of the subject matter described in this disclosure can be implemented in a micro-feature device. The micro-feature device can include a substantially rigid transparent substrate. The device can include a plurality of macrowells defined in the transparent substrate. Each macrowell can have a width in the range of about one millimeter to about 35 millimeters and a depth in the range of about two millimeters to about 12 millimeters. Each macrowell can include a respective plurality of microwells defined in a respective lower surface of the macrowell. Each microwell can have a width in the range of about 50 microns to about 500 microns and a depth in the range of about 50 microns to about 500 microns.

In some implementations, each pair of adjacent macrowells can be separated by a distance in the range of about 0.5 millimeters to about five millimeters. In some implementations, each macrowell can have a height-to-width ratio in the range of about 1:1 to about 5:1. In some implementations, each pair of adjacent microwells can be separated by a distance in the range of about 50 microns to about 200 microns. In some implementations, each microwell can have a height-to-width ratio in the range of about 1:1 to about 5:1.

In some implementations, the transparent substrate can include at least one of polystyrene, polycarbonate, polymethylpentene, a cyclic olefin copolymer, and polydimethylsiloxane (PDMS). In some implementations, a distance between a lower surface of the transparent substrate and a lower surface of at least one microwell of the plurality of microwells can be between about 25 microns and about 200 microns. In some implementations, a distance between a lower surface of the transparent substrate and a lower surface of each microwell of the plurality of microwells can be substantially uniform.

In some implementations, a bottom surface of each microwell can include a layer of polymer material. In some implementations, the layer of polymer material at the bottom of each microwell can have a thickness in the range of about 0.5 microns to about 25 microns. In some implementations, the layer of polymer material can include at least one of polyurethane and epoxy. In some implementations, the layer of polymer material can include silicone, such as PDMS.

Another innovative aspect of the subject matter described in this disclosure can be implemented in a method of fabricating a micro-feature device. The method can include positioning an outer mold in contact with at least two opposing side surfaces and a portion of an upper surface of a substantially rigid transparent substrate. A coefficient of thermal expansion of the outer mold can be less than a coefficient of thermal expansion of the transparent substrate. The method can include positioning an inner mold on the upper surface of the transparent substrate. The coefficient of thermal expansion of the transparent substrate can be less than a coefficient of thermal expansion of the inner mold. The method can include applying a first platen at a first temperature to the inner mold to cause the inner mold to form a plurality of macrowells defined in the transparent substrate, and a respective plurality of microwells defined in a respective lower surface of each macrowell. Each macrowell can have a width in the range of about one millimeter to about 35 millimeters and a depth in the range of about two millimeters to about 12 millimeters. Each microwell can have a width in the range of about 50 microns to about 500 microns and a depth in the range of about 50 microns to about 500 microns. The method can include cooling the outer mold, the transparent substrate, and the inner mold to a second temperature, lower than the first temperature. The method can include removing the inner mold from the transparent substrate. The method can include removing the transparent substrate from the outer mold.

In some implementations, the method can include selecting a first material for the inner mold. The first material can be selected to be softer than the transparent substrate. The first material can have a durometer measurement in the range of about 30A to about 80A. In some implementations, the method can include selecting a second material for the outer mold. The second material can be selected to be harder than the transparent substrate. The second material can have a Mohs hardness in the range of about 2 to about 3 or a Rockwell hardness in the range of about R120 to about R130. In some implementations, the inner mold is formed from polydimethylsiloxane (PDMS).

In some implementations, the inner mold can include a plurality of macropillars extending outward from a surface of the inner mold. Each macropillar can include a respective plurality of micropillars extending outwards from a respective surface of the macropillar. In some implementations, each macropillar can have a width in the range of about one millimeter to about 35 millimeters and a height in the range of about two millimeters to about 12 millimeters. In some implementations, each pair of adjacent macropillars can be separated by a distance in the range of about 0.5 millimeters to about five millimeters. In some implementations, each macropillar can have a height-to-width ratio in the range of about 1:1 to about 5:1. In some implementations, each micropillar can have a width in the range of about 50 microns to about 500 microns and a height in the range of about 50 microns to about 500 microns. In some implementations, each pair of adjacent micropillars can be separated by a distance in the range of about 25 microns to about 200 microns. In some implementations, each micropillar can have a height-to-width ratio in the range of about 1:1 to about 5:1.

In some implementations, the inner mold can include a material that is permeable to gas. In some implementations, the transparent substrate can be formed from at least one of polystyrene, polycarbonate, polymethylpentene, and a cyclic olefin copolymer.

In some implementations, the method can include providing a supporting platform in contact with a lower surface of the transparent substrate and applying a second platen at a second temperature to the supporting platform. In some implementations, the method can include providing a supporting platform including a thermal buffer layer in contact with a lower surface of the transparent substrate and applying a second platen at an ambient temperature to the supporting platform. In some implementations, the thermal buffer layer can be formed from one of polyether ether ketone (PEEK) or a polyimide film.

In some implementations, the thermal buffer layer can be formed from one of polyether ether ketone (PEEK) or a polyimide film. In some implementations, the method can include depositing a coating comprising a polymer material on a bottom surface of each microwell. In some implementations, the coating can include at least one of polyurethane and epoxy. In some implementations, the coating can include silicone, such as PDMS. In some implementations, the coating also can include a solvent. In some implementations, the coating can be a conformal coating applied over the bottom surface and sidewalls of each microwell. In some implementations, the coating can have a thickness in the range of about 0.5 microns to about 25 microns.

Another innovative aspect of the subject matter described in this disclosure can be implemented in a method of fabricating a micro-feature device. The method can include positioning an outer mold in contact with at least two opposing side surfaces and a portion of an upper surface of a substantially rigid transparent substrate. A coefficient of thermal expansion of the outer mold can be less than a coefficient of thermal expansion of the transparent substrate. The method can include positioning a first inner mold on the upper surface of the transparent substrate. The coefficient of thermal expansion of the transparent substrate can be less than a coefficient of thermal expansion of the first inner mold. The method can include applying a first platen at a first temperature to the first inner mold to cause the first inner mold to form a plurality of macrowells defined in the transparent substrate. Each macrowell can have a width in the range of about one millimeter to about 35 millimeters and a depth in the range of about two millimeters to about 12 millimeters. The method can include cooling the outer mold, the transparent substrate, and the first inner mold to a second temperature, lower than the first temperature. The method can include removing the first inner mold from the transparent substrate. The method can include depositing a layer of polymer material onto a bottom surface of each macrowell. The method can include positioning a second inner mold on an upper surface of the layer of polymer material to cause the second inner mold to form a respective plurality of microwells defined in a respective lower surface of each macrowell. Each microwell can have a width in the range of about 50 microns to about 500 microns and a depth in the range of about 50 microns to about 500 microns. The method can include curing the layer of polymer material.

In some implementations, the layer of polymer material at the bottom of each microwell can have a thickness in the range of about 0.5 microns to about 25 microns. In some implementations, the second inner mold can include at least one of polyurethane and epoxy. In some implementations, the second inner mold can include an elastomer, such as PDMS or fluorinated PDMS. In some implementations, the second inner mold can include a material that is permeable to gas. In some implementations, the layer of polymer material can include at least one of polyurethane and epoxy. In some implementations, the layer of polymer material can include silicone, such as PDMS.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar elements, and/or structurally similar elements throughout the various drawings. The drawings are not necessarily to scale. Instead, emphasis is placed upon illustrating the principles of the teachings. The drawings are not intended to limit the scope of the present teachings in any way. The system and method may be better understood from the following illustrative description with reference to the following drawings in which:

FIG. 1A shows a perspective view of a micro-feature device, according to an illustrative implementation.

FIG. 1B shows a top view of the micro-feature device shown in FIG. 1A, according to an illustrative implementation.

FIG. 1C shows a cross-sectional view of the micro-feature device shown in FIG. 1B along the line labeled A-A′, according to an illustrative implementation.

FIG. 2 shows a flow chart for a process of manufacturing a micro-feature device, according to an illustrative implementation.

FIG. 3A shows a rigid mold that can be used in the manufacturing process of FIG. 2, according to an illustrative implementation.

FIG. 3B shows a soft mold that can be used in the manufacturing process of FIG. 2, according to an illustrative implementation.

FIG. 3C shows an enlarged view of a section of the soft mold shown in FIG. 3B, according to an illustrative implementation.

FIGS. 4A-4P show cross-sectional views of example stages of construction of a micro-feature device according to the manufacturing process shown in FIG. 2.

FIG. 5A shows a photograph of a micro-feature device that can be fabricated according to the manufacturing process shown in FIG. 2, according to an illustrative implementation.

FIG. 5B shows an enlarged view of a portion of the micro-feature device shown in FIG. 5A, according to an illustrative implementation.

FIGS. 6A and 6B show images of cell cultures within a microwell of the micro-feature device, according to an illustrative implementation.

FIG. 7 shows a flow chart for a process of manufacturing a micro-feature device, according to an illustrative implementation.

FIGS. 8A-8L show cross-sectional views of example stages of construction of a micro-feature device according to the manufacturing process shown in FIG. 7.

Like reference numbers and designations in the various drawings indicate like elements.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The systems and methods disclosed are generally related to a micro-feature device. More particularly, the systems and methods enable fabrication of a device that can be used to culture cells for high-throughput screening. In some implementations, the device is used to model how human organ systems respond to agents such as toxins or medications. In some implementations, the cell cultures can be imaged using a high resolution microscope at high magnification, such as 100×.

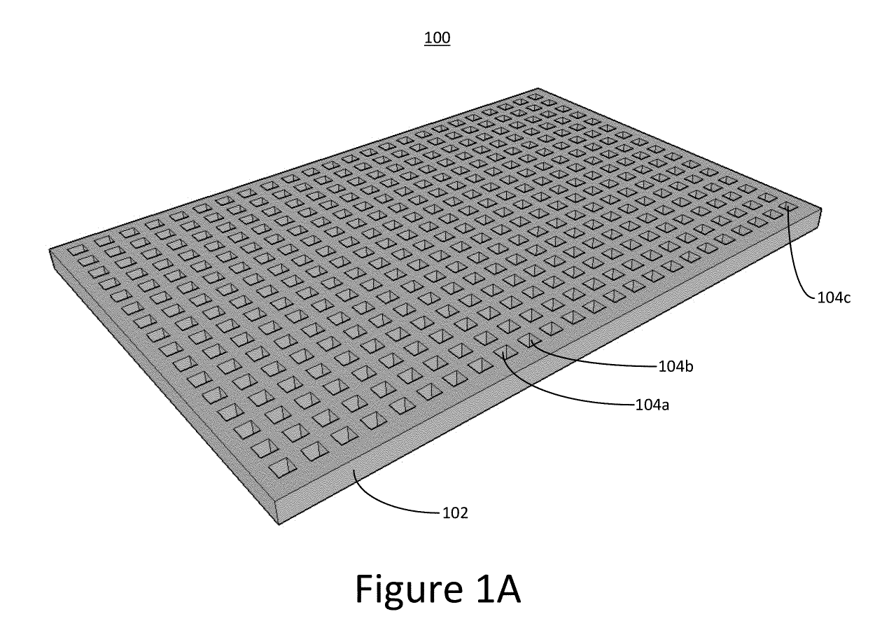

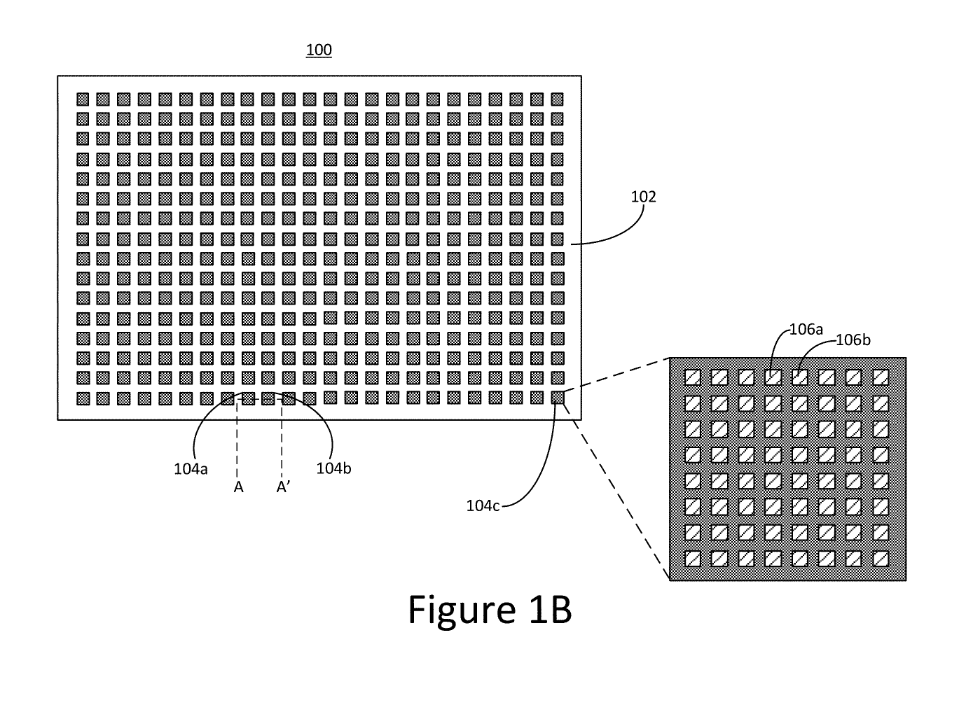

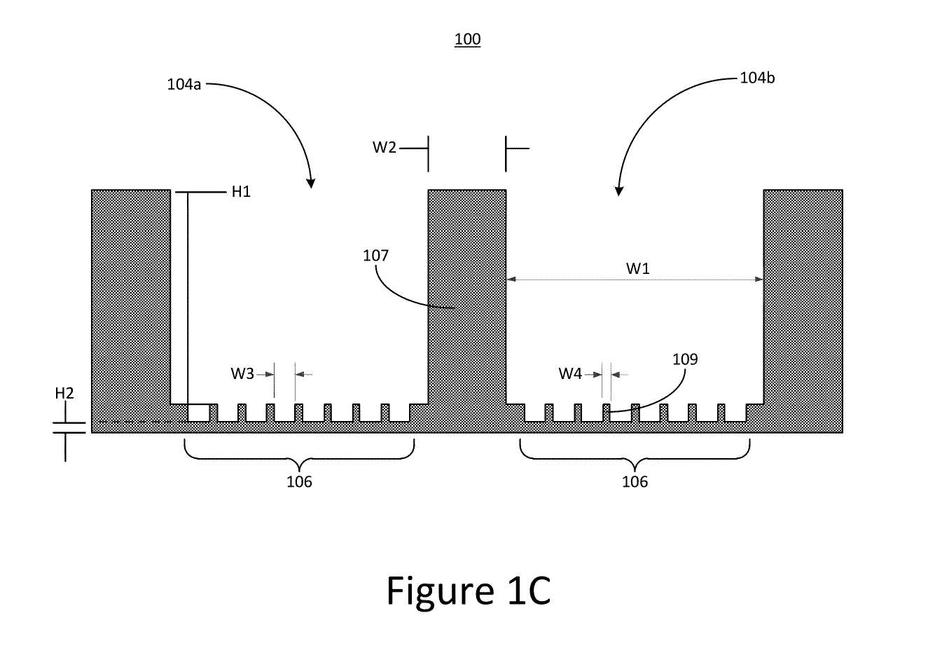

FIG. 1A shows a perspective view of a micro-feature device 100, according to an illustrative implementation. The micro-feature device 100 includes a substrate 102 in which a plurality of macrowells, such as the macrowells 104a, 104b, and 104c (generally referred to as macrowells 104) are defined. For illustrative purposes, not all of the macrowells 104 are labeled in FIG. 1A. The micro-feature device 100 is shown with a total of 384 macrowells 104, arranged in a 16×24 grid. However, in other implementations, the micro-feature device 100 can include a different total number or arrangement of macrowells 104. For example, the micro-feature device can include 6, 24, 96, or 1536 macrowells 104. In some implementations, the macrowells 104 can be arranged on the substrate 102 in a 2:3 rectangular grid. In other implementations, the macrowells 104 can be arranged in a rectangular grid having a different aspect ratio, or in a non-rectangular array.

Each macrowell 104 has a substantially square cross-sectional shape and is defined by four sidewalls and a bottom. In some other implementations, the macrowells 104 can have other cross-sectional shapes. For example, the macrowells 104 can have polygonal cross-sectional shapes, such as triangular, rectangular, or hexagonal cross-sectional shapes. In some other implementations, the macrowells 104 can have circular cross-sectional shapes. In still other implementations, the macrowells 104 can have polygonal cross-sectional shapes with rounded corners. The top of each macrowell 104 remains open.

FIG. 1B shows a top view of the micro-feature device 100 shown in FIG. 1A, according to an illustrative implementation. Also shown in FIG. 1B an enlarged top view of the macrowell 104c. As shown in the enlarged view, the macrowell 104c includes a plurality of microwells, such as the microwells 106a and 106b (generally referred to as microwells 106). The microwells 106 are defined in a bottom surface of the macrowell 104c. FIG. 1B shows the macrowell 104c including 64 total microwells arranged in an 8×8 grid, however, in other implementations, the macrowell 104c can include a different total number or arrangement of microwells 106c. For example, in some implementations the macrowell 104c can include between 10 and 50 microwells 106, between 50 and 100 microwells 106, between 100 and 150 microwells 106, between 150 and 200 microwells 106, between 200 and 250 microwells 106, between 250 and 300 microwells 106, between 300 and 350 microwells 106, or between 350 and 400 microwells 106. In some implementations, the macrowell 104c can include more than 400 microwells 106. The arrangement of the microwells 106 within the macrowell 104c can be selected based on the shape of the macrowell 104c. For example, as shown in FIG. 1B, a square grid of microwells 106 may be appropriate when the macrowell has a square cross-sectional shape. In implementations in which the macrowell 104c has a different shape, the arrangement of the microwells 106 within the macrowell may be adjusted. For example, if the macrowell 104c has a hexagonal cross-sectional shape, the microwells 106 could be arranged in a generally hexagonal array within the macrowell 104. In other implementations, the arrangement of the microwells may be independent of the cross-sectional shape of the macrowell 104c. For example, the macrowell 104c may have a circular cross-sectional shape, and the microwells 106 may be arranged in a rectangular grid within the macrowell 104c. The microwells 106 are enclosed by sidewalls and a bottom surface, but the top of each microwell 106 remains open. Each of the other macrowells 104 also can include a plurality of microwells 106 defined in a bottom surface, similar to those shown in the enlarged view of the macrowell 104c.

In some implementations, the micro-feature device 100 can serve as a model for an organ, for example by allowing cells to be cultured within each microwell 106. In some implementations, the small enclosed space defined by each microwell 106 can provide a favorable environment in which to culture human hepatocytes. Thus, the micro-feature device 100 can serve as a model for the human liver. In some implementations, hepatocytes can be cultured in the microwells 106 for relatively long time periods. For example, primary hepatocyte characteristics can be maintained within the microwells 106 over a period of at least about three weeks to about five weeks. This can allow the micro-feature device 100 to be used to test the efficacy of drugs on diseases that impact liver cells, such as malaria, over a relatively long period of time.

In some implementations, the arrangement of macrowells 104 and microwells 106 in the micro-feature device 100 can facilitate high-throughput screening of drugs in a laboratory setting. For example, each microwell 106 defines an enclosed space that can contain a respective cell culture, but also remains open on top. Likewise, the macrowells 104 in which the microwells 106 are defined also remain open on top. Thus, cell samples can be easily introduced into the microwells for culturing, for example via pipettes. In some implementations, reagents such as nutrients, toxins, drugs, or other biologically active materials can be introduced into all of the microwells 106 of a single macrowell 104 substantially simultaneously, due to the arrangement of microwells 106 within the macrowells 104. For example, the microwells 106 each have substantially the same dimensions and are distributed substantially uniformly over the entire bottom surface of each macrowell 104. Thus, large reagent samples can be introduced into a macrowell 104 and an approximately equal portion of the reagent can flow into each microwell 106. The arrangement of the macrowells 104 also can facilitate high-throughput screening. For example, the grid pattern of macrowells 104 can easily interface with standard microtiter equipment, such as robotic pipetters and imaging equipment.

FIG. 1C shows a cross-sectional view of the micro-feature device 100 shown in FIG. 1B along the line labeled A-A′, according to an illustrative implementation. Two macrowells 104a and 104b are shown. Each macrowell 104 includes a respective row of eight microwells 106 defined in its lower surface. The macrowells 104 have a width W1 and a height H1. The macrowell 104a is separated from the macrowell 104b by a sidewall 107 having a width W2. In some implementations, the width W1 of the macrowells 104 can be in the range of about one millimeter to about five millimeters. For example, the width W1 of the macrowells 104 can be about one millimeter, about two millimeters, about three millimeters, about four millimeters, about five millimeters, about 10 millimeters, about 15 millimeters, about 20 millimeters, about 25 millimeters, or about 30 millimeters. In some implementations, the height H1 of the macrowells 104 can be in the range of about two millimeters to about 12 millimeters. For example, the width W1 of the macrowells 104 can be about two millimeters, about three millimeters, about four millimeters, about five millimeters, about 8 millimeters, about 10 millimeters, or about 12 millimeters. In some implementations, the height-to-width ratio for the macrowells 104 can be in the range of about 1:1 to about 5:1. For example, the height-to-width ratio for the macrowells 104 can be about 1:1, about 2:1, about 3:1, about 4:1, about 5:2, or about 5:1. In some other implementations, the height-to-width ratio for the macrowells 104 can be less than 1:1. For example, the height-to-width ratio for the macrowells 104 can be about 3:5. The width W2 of the sidewall 107 separating the macrowells 104 can be in the range of about 0.5 millimeters to about five millimeters. For example, the width W2 of the sidewall 107 can be about 0.5 millimeters, about one millimeter, about two millimeters, about three millimeters, about four millimeters, or about five millimeters.

The microwells 106 have a width W3, and each microwell is separated from an adjacent microwell by a sidewall 109 having a width W4. In some implementations, the width W3 of the microwells 106 can be in the range of about 50 microns to about 500 microns. For example, the width W3 of the microwells 106 can be about 50 microns, about 100 microns, about 200 microns, about 300 microns, about 400 microns, or about 500 microns. In some implementations, the height of the microwells 106 can be in the range of about 50 microns to about 500 microns. For example, the height of the microwells 106 can be about 50 microns, about 100 microns, about 200 microns, about 300 microns, about 400 microns, or about 500 microns. In some implementations, the height-to-width ratio for the microwells 106 can be in the range of about 1:1 to about 5:1. For example, the height-to-width ratio for the microwells 106 can be about 1:1, about 2:1, about 3:1, about 4:1, or about 5:1. In some other implementations, the height-to-width ratio for the microwells 106 can be less than 1:1.

The micro-feature device 100 has a thickness H2 between the bottom surface of the micro-feature device 100 and the bottom surface of each microwell 106. In some implementations, the micro-feature device 100 can be used to image respective cell cultures within the microwells 106. Therefore, the material used to form the micro-feature device can be selected to be optically clear, so that cell cultures can be imaged by equipment positioned outside of the micro-feature device 100. For example, high resolution microscopes can be used to view cell cultures through the bottom surface of the micro-feature device 100. Thus the thickness H2 between the bottom surface of the micro-feature device 100 and the bottom surface of each microwell 106 can be selected to facilitate high resolution microscopy (e.g., 100× magnification). In some implementations, microscope objectives may have very short focal lengths, and therefore the thickness H2 should be relatively small in order to position a microscope outside the micro-feature device 100 within a desired focal length of the bottom of each microwell 106. In some implementations, the thickness H2 can be in the range of about 25 microns to about 200 microns. In addition, to facilitate accurate imaging of cells in all of the microwells 106 of the micro-feature device 100, the thickness H2 can be substantially uniform across the entire micro-feature device 100.

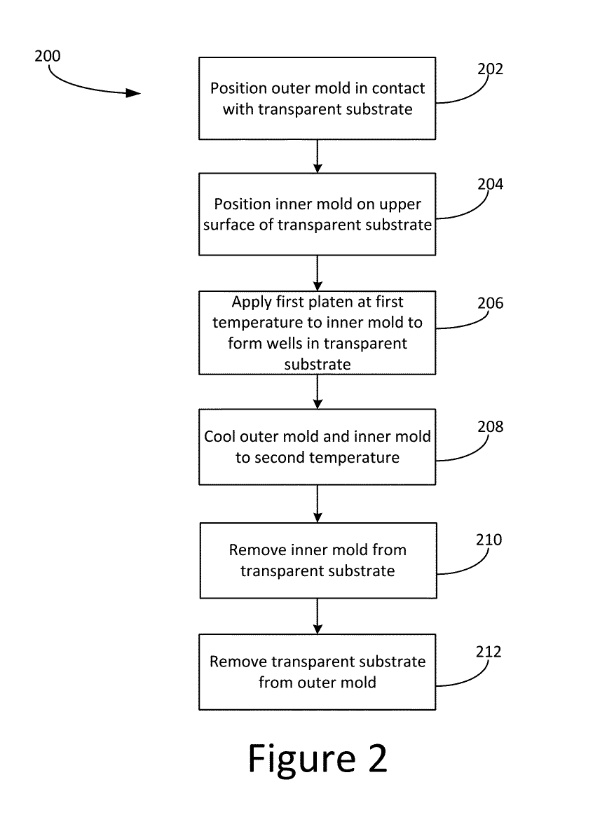

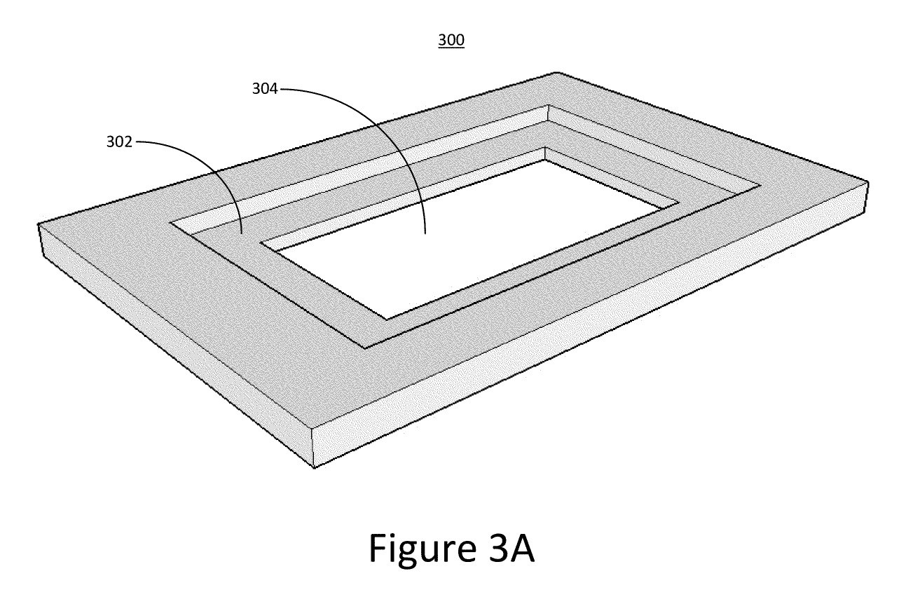

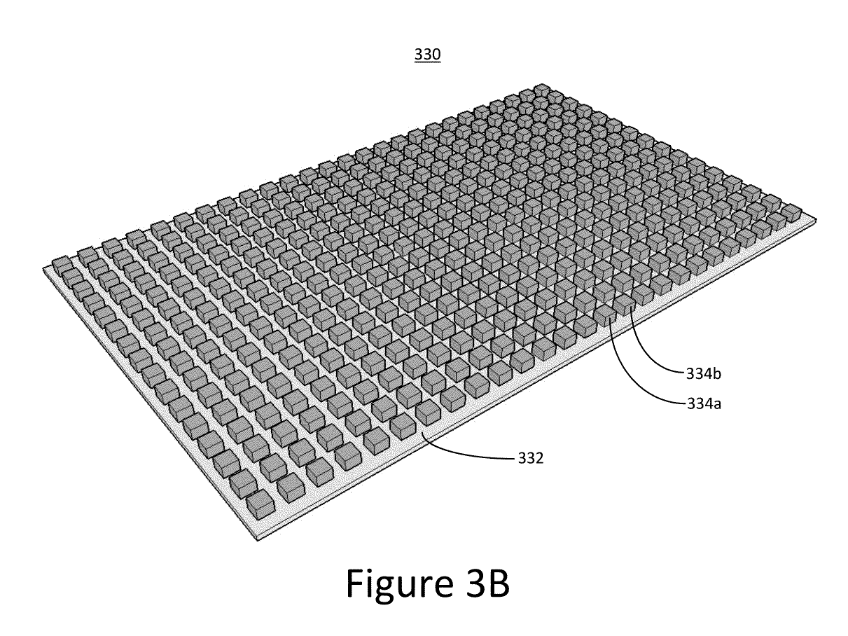

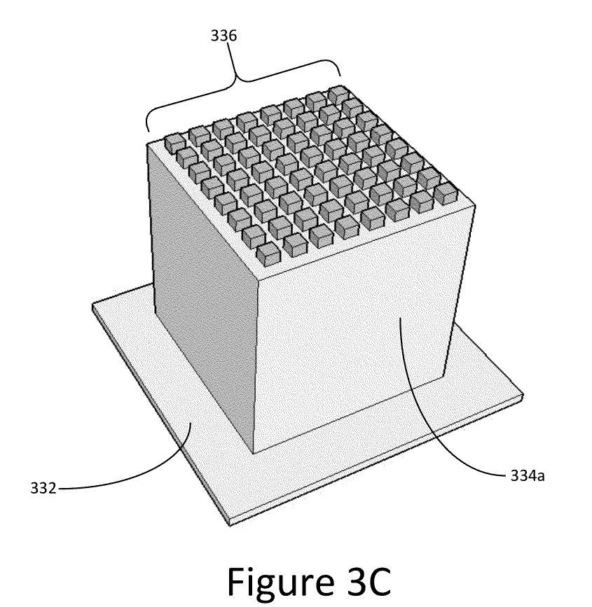

FIG. 2 shows a flow chart for a process 200 of manufacturing a micro-feature device, according to an illustrative implementation. In brief overview, the process 200 includes positioning an outer mold in contact with a transparent substrate (stage 202). The process 200 includes positioning an inner mold on an upper surface of the transparent substrate (stage 204). The process 200 includes applying a first platen at a first temperature to the inner mold to form a plurality of macrowells and microwells defined in the transparent substrate (stage 206). The process 200 includes cooling the outer mold and the inner mold to a second temperature (stage 208). The process 200 includes removing the inner mold from the transparent substrate (stage 210). The process 200 includes removing the transparent substrate from the outer mold (stage 212). The process 200 is described in greater detail below in connection with FIGS. 3A-3C and FIGS. 4A-40.

Referring now to FIG. 3A, an outer mold 300 that can be used in the manufacturing process 200 of FIG. 2 is shown, according to an illustrative implementation. The outer mold 300 is generally rectangular in shape, and includes a recess 302 and an cutout 304 formed in the recess 302. For illustrative purposes, what will be referred to as the bottom of the outer mold 300 is shown facing up in FIG. 3A.

The material used to form the outer mold 300 also can be selected to have a relatively low coefficient of thermal expansion, so that the outer mold 300 will not expand significantly when subjected to high temperatures. In some implementations, the material used to form the outer mold 300 can have a coefficient of thermal expansion in the range of about 12 (10⁻⁶ m/(m K)) to about 56 (10⁻⁶ m/(m K)). For example, in some implementations, the material used to form the outer mold 300 can have a coefficient of thermal expansion of about 12 (10⁻⁶ m/(m K)), about 22.2 (10⁻⁶ m/(m K)), or about 55.8 (10⁻⁶ m/(m K)). In some implementations, the material for the outer mold 300 is selected to have a coefficient of thermal expansion that is lower than the coefficient of thermal expansion of a substrate that is to be formed into the micro-feature device through a molding process. This can facilitate removing the manufactured micro-feature device from the outer mold at the end of the molding process, as discussed further below in connection with the process 200 shown in FIG. 2.

The material used to form the outer mold 300 also can be selected to have a high thermal conductivity, so that heat from a platen applied to the outer mold 300 can be efficiently transferred to the substrate. In some implementations, the outer mold 300 is formed from a relatively rigid material. For example, the outer mold 300 can be formed from a metal, such as aluminum. In other implementations, the outer mold 300 can be formed from a metal alloy. Metals can be well suited for use in the outer mold 300, because many metals have relatively low coefficients of thermal expansion, relatively high thermal conductivities, and also are structurally rigid. However, in some other implementations, the outer mold 300 can be formed from a semiconducting material such as silicon, or a rigid plastic material such as polyetherimide (PEI) or polysulfone, provided that the selected material has a suitable low coefficient of thermal expansion and a suitably high thermal conductivity. In implementations in which the outer mold 300 is formed from a metal or metal alloy, the outer mold can have a Mohs hardness in the range of about 2 to about 3. In implementations in which the outer mold 300 is formed from a polymer, the outer mold can have a Rockwell hardness in the range of about R120 to about R130.

In some implementations, the recess 302 and the cutout 304 can be formed in the outer mold 304 using conventional machining techniques. The size and shape of the recess 302 of the outer mold 300 can be selected based on the size and shape of the substrate that is to be molded to form the micro-feature device. Thus, the height of the sidewalls of the recess 302 can be selected to be approximately equal to the thickness of the substrate. In some implementations, the substrate (and therefore the sidewalls of the recess 302) can have a thickness in the range of about 1 millimeter to about 5 millimeters. For example, the substrate can have a thickness of about 1 millimeter, about 2 millimeters, about 3 millimeters, about 4 millimeters, or about 5 millimeters. As discussed above, the bottom surface of the outer mold 300 is shown facing up in FIG. 3A. During the manufacturing process, the outer mold 300 can be turned over and positioned above the substrate, with the substrate positioned inside the recess 302. The sidewalls of the recess 302 can be in contact with the substrate and the horizontal surface of the recess 302 can be in contact with a portion of the top surface of the substrate, such that the substrate is held in place by the outer mold 300. The cutout 304 can provide a space through which an inner mold, described below in connection with FIG. 3B and FIG. 3C, can contact the upper surface of the substrate during the molding process.

FIG. 3B shows an inner mold 330 that can be used in the manufacturing process 200 of FIG. 2, according to an illustrative implementation. FIG. 3C shows an enlarged view of a section of the inner mold 330 shown in FIG. 3B, according to an illustrative implementation. As shown in FIG. 3B, the inner mold 330 includes a substantially planar portion and a plurality of macropillars, such as the macropillars 334a and 334b (generally referred to as macropillars 334). For illustrative purposes, not all of the macropillars 334 are labeled in FIG. 3B. As shown in FIG. 3C, the macropillar 334 includes a plurality of micropillars 336 projecting outward from its upper surface. Each macropillar 334 can include a respective plurality of micropillars 336. For clarity, the micropillars 336 are not shown, due to their small size, in the view of the inner mold 330 shown in FIG. 3B.

As described further below, the inner mold 330 can be pressed into a substrate in order to mold the substrate into the shape of the micro-feature device 100 shown in FIGS. 1A-1C. The macropillars 334 and micropillars 336 can press into the substrate to form spaces corresponding to the macrowells 104 and microwells 106 shown in FIG. 1B and FIG. 1C. Thus, the dimensions of the macropillars 334 can be substantially the same as the dimensions of the macrowells 104 described above, and the dimensions of the micropillars 334 can be substantially the same as the dimensions of the microwells 104. For example, the micro-feature device can include 6, 24, 96, 384, or 1536 macropillars 334. In some implementations, the macropillars 334 can be arranged on the surface of the inner mold 330 in a grid pattern having an aspect ratio of 2:3. In other implementations, the macropillars 334 can be arranged in a rectangular grid having a different aspect ratio, or in a non-rectangular array.

Similar to the macrowells discussed above, the width of the macropillars 334 can be in the range of about one millimeter to about 35 millimeters. For example, the width of the macropillars 334 can be about one millimeter, about two millimeters, about three millimeters, about four millimeters, about five millimeters, about 10 millimeters, about 15 millimeters, about 20 millimeters, about 25 millimeters, or about 30 millimeters. In some implementations, the height of the macropillars 334 can be in the range of about two millimeters to about 12 millimeters. For example, the width of the macropillars 334 can be about two millimeter, about three millimeters, about four millimeters, about five millimeters, about 8 millimeters, about 10 millimeters, or about 12 millimeters. In some implementations, the height-to-width ratio for the macropillars 334 can be in the range of about 1:1 to about 5:1. For example, the height-to-width ratio for the macropillars 334 can be about 1:1, about 2:1, about 3:1, about 4:1, about 5:2, or about 5:1. In some other implementations, the height-to-width ratio for the macropillars 334 can be less than 1:1. For example, the height-to-width ratio for the macropillars 334 can be about 3:5. The macropillars 334 can be separated by a distance in the range of about 0.5 millimeters to about five millimeters. For example, the macropillars 334 can be separated by a distance of about one millimeter, about two millimeters, about three millimeters, about four millimeters, or about five millimeters.

Similarly, the arrangement of the micropillars 336 on the surface of the macropillar 334a can be the same as the arrangement of the microwells within a macrowell. For example, in some implementations the macropillar 334a can include between 10 and 50 micropillars 336, between 50 and 100 micropillars 336, between 100 and 150 micropillars 336, between 150 and 200 micropillars 336, between 200 and 250 micropillars 336, between 250 and 300 micropillars 336, between 300 and 350 micropillars 336, or between 350 and 400 micropillars 336. In some implementations, the macropillar 334a can include more than 400 micropillars 336. The arrangement of the micropillars 336 on the surface of the macropillar 334a can be selected based on the shape of the macropillar 334a. For example, as shown in FIG. 1B, a square grid of micropillars 336 may be appropriate when the macropillar 334a has a square cross-sectional shape. In implementations in which the macropillar 334a has a different shape, the arrangement of the micropillars 336 on the surface of the macropillar 334a may be adjusted. For example, if the macropillar 334a has a hexagonal cross-sectional shape, the micropillars 336 could be arranged in a generally hexagonal array on the surface of the macropillar 334a. In other implementations, the arrangement of the micropillars 336 may be independent of the cross-sectional shape of the macropillar 334a. For example, the macropillar 334a may have a circular cross-sectional shape, and the micropillars 336 may be arranged in a rectangular grid on the surface of the macropillar 334a.

In some implementations, the width of the micropillars 336 can be in the range of about 50 microns to about 500 microns. For example, the width of the micropillars 336 can be about 50 microns, about 100 microns, about 200 microns, about 300 microns, about 400 microns, or about 500 microns. In some implementations, the height of the micropillars 336 can be in the range of about 50 microns to about 500 microns. For example, the height of the micropillars 336 can be about 50 microns, about 100 microns, about 200 microns, about 300 microns, about 400 microns, or about 500 microns. In some implementations, the height-to-width ratio for the micropillars 336 can be in the range of about 1:1 to about 5:1. For example, the height-to-width ratio for the micropillars 336 can be about 1:1, about 2:1, about 3:1, about 4:1, or about 5:1. In some other implementations, the height-to-width ratio for the micropillars 336 can be less than 1:1.

In some implementations, the inner mold 330 is formed from a relatively soft material. For example, the material used to form the inner mold 330 can have a durometer measurement in the range of about 30A to about 80A. In some implementations, the material used to form the inner mold 330 can have a durometer measurement of about 50A. In some implementations, the soft material is a polymer. For example, the inner mold 330 can be formed from polydimethylsiloxane (PDMS). In some other implementations, the inner mold 330 can be formed from a soft silicone or a rubber material. In some other implementations, the inner mold 330 can be formed from two materials. For example, the inner mold 330 can include a relatively rigid material forming a relatively thin layer including the macropillars 334 and the micropillars 336, and a relatively soft material forming the rest of the inner mold 330. In some other implementations, the inner mold 330 can include a relatively soft material including the macropillars 334 and the micropillars 336, and a relatively rigid material forming the rest of the inner mold 330. The relatively rigid material forming the rest of the inner mold 330 could also be used to form supporting structures inside the macropillars.

The material used to form the inner mold 330 also can be selected to have a relatively high coefficient of thermal expansion, so that the inner mold will tend to contract significantly after being cooled from a high temperature to room temperature. In some implementations, the material for the inner mold 330 can be selected to have relatively high gas permeability, and to have a coefficient of thermal expansion that is higher than the coefficient of thermal expansion of the substrate that is to be formed into the micro-feature device. This can help to avoid bubble-like defects in the manufactured micro-feature device and can facilitate removing the inner mold 330 from the manufactured micro-feature device, as discussed further below in connection with the process 200 shown in FIG. 2. For example, in some implementations, the material used to form the inner mold 330 can have a coefficient of thermal expansion in the range of about 300 (10⁻⁶ m/(m K)) to about 350 (10⁻⁶ m/(m K)). For example, in some implementations, the material used to form the inner mold 330 can have a coefficient of thermal expansion of about 310 (10⁻⁶ m/(m K)). In some implementations, the material used to form the inner mold 330 can have a permeability in the range of about 100 Barrer to about 800 Barrer for nitrogen and a permeability in the range of about 200 Barrer to about 1200 Barrer for oxygen. In some implementations, the material used to form the inner mold 330 can have a permeability of about 400 for nitrogen and about 800 for oxygen.

In some implementations, the inner mold 330 can be fabricated using a rigid master mold. For example, in some implementations, a mold made from a rigid material may be more durable and longer lasting than one made from a soft material. However, rigid materials typically do not have the high coefficients of thermal expansion and flexibility that are desirable for the inner mold 330. Moreover, a rigid mold may take longer to fabricate than a soft mold, increasing the required turnaround time for prototyping. As a result, an approach that combines durability of rigid molds with the rapid fabrication and ease of de-molding of soft molds, can be advantageous. Thus, a rigid master mold can be fabricated with small features corresponding to the micropillars 336. Such small features can be formed in a rigid master mold using various techniques, such as micromachining or lithography, which may not be suitable for defining features in the softer material to be used as the inner mold. The shape of the rigid master mold can then be transferred to the inner mold, for example by injection or replica molding. In some implementations, the master mold can be formed from silicon or a metal such as aluminum or nickel.

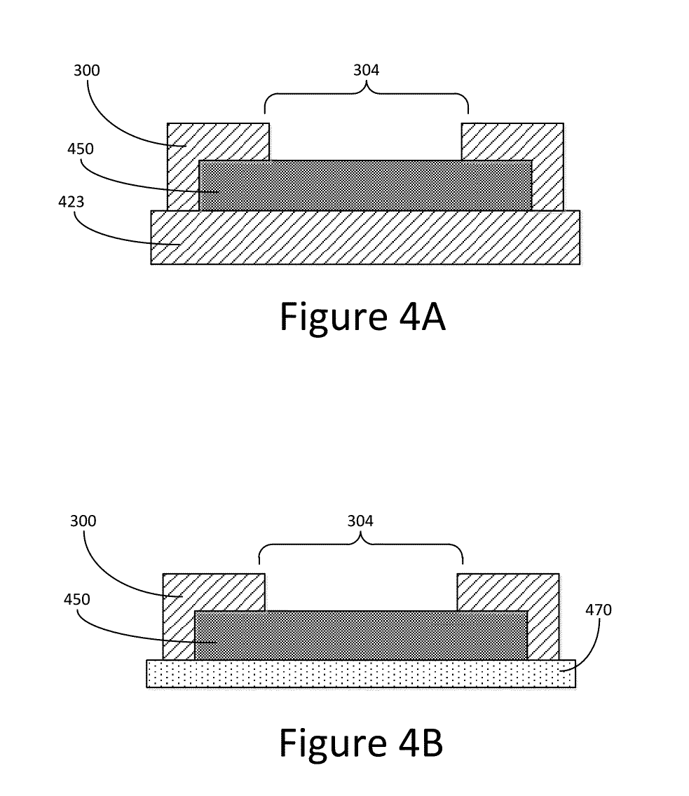

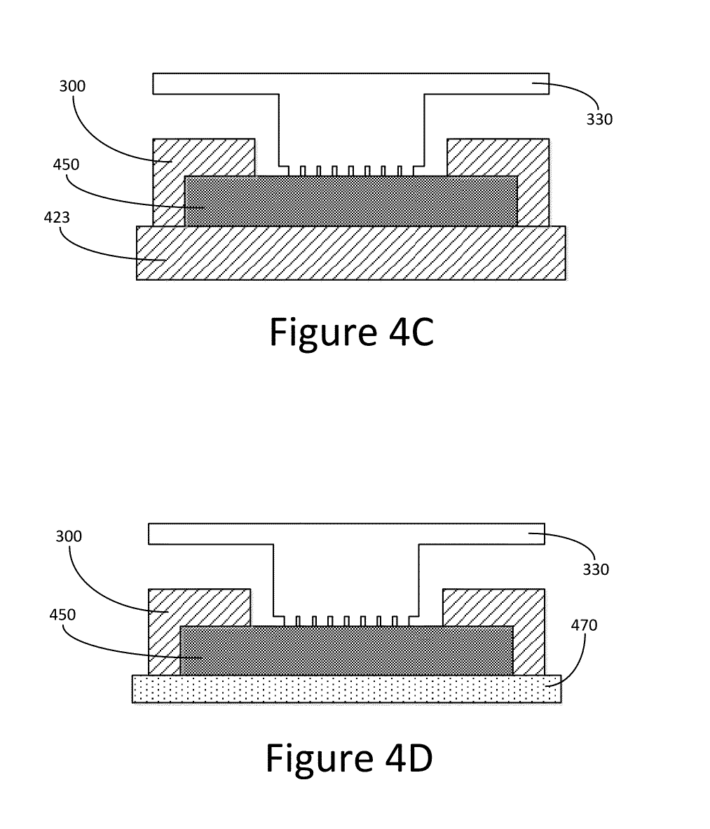

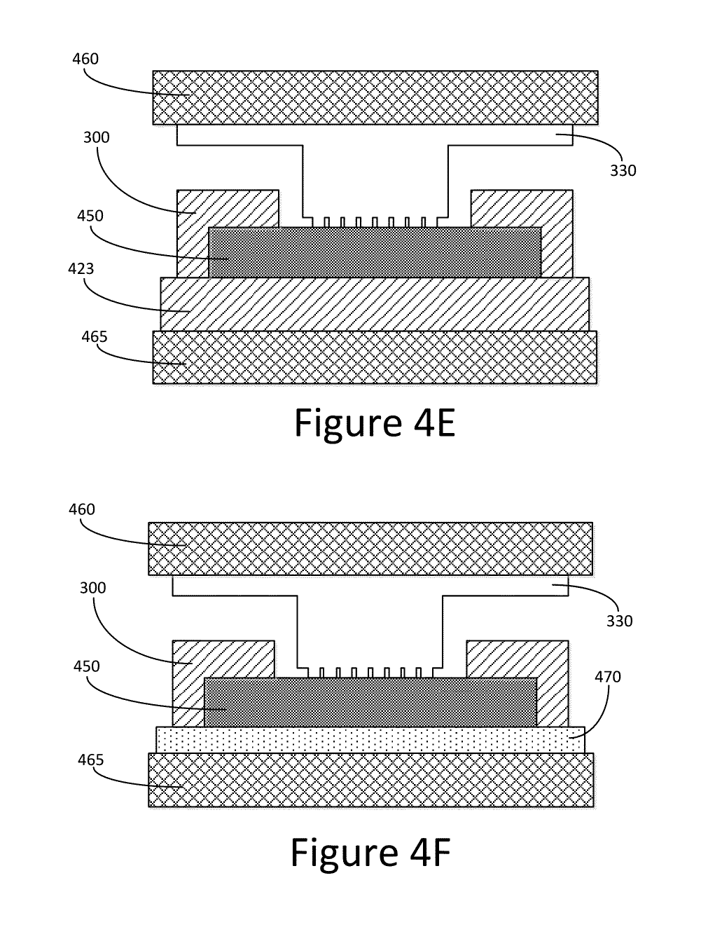

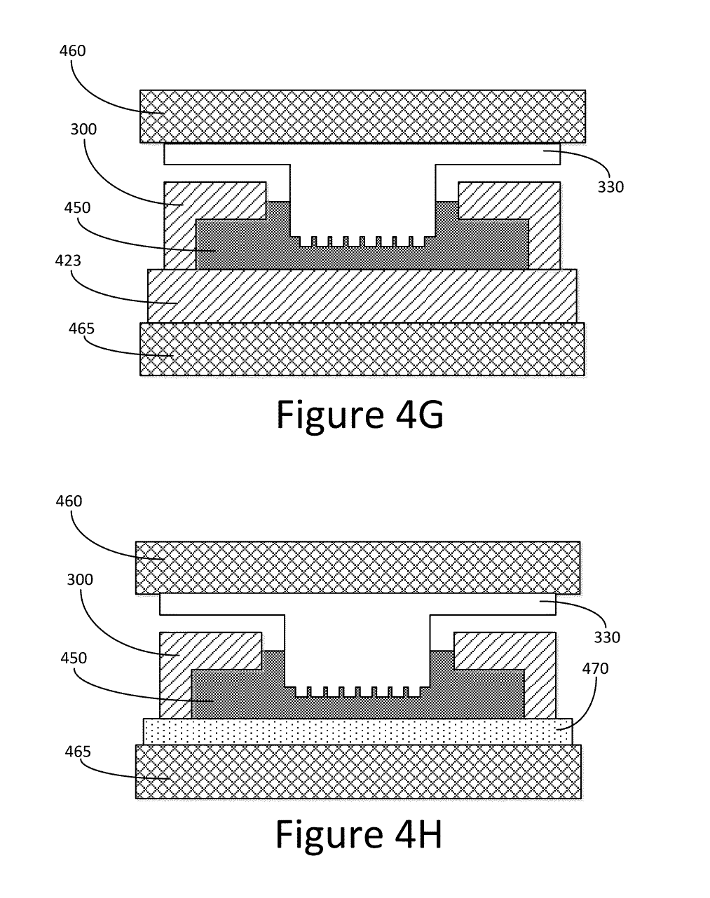

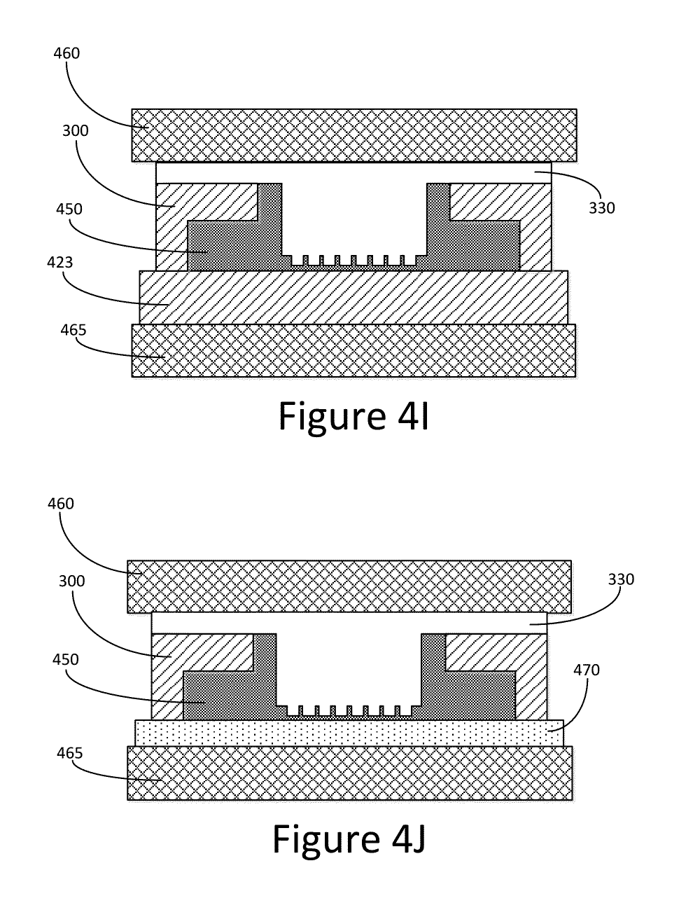

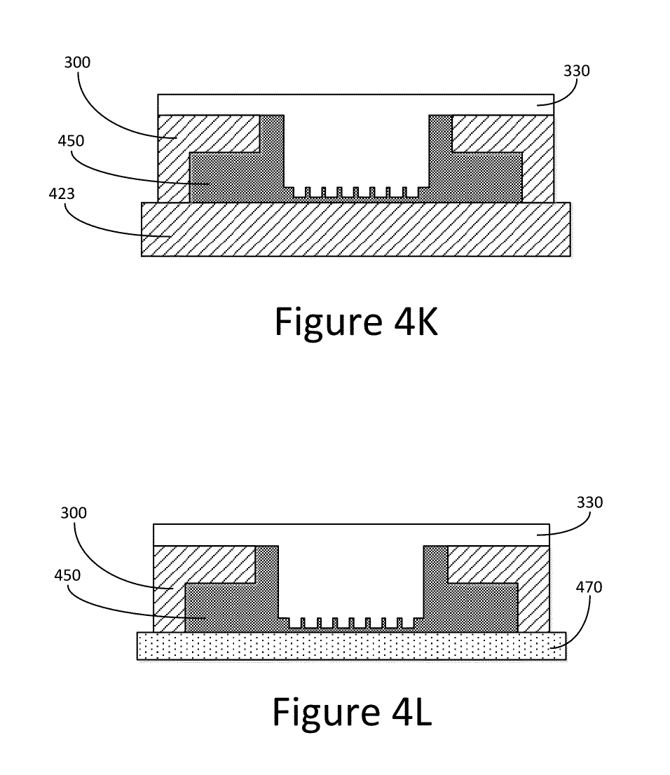

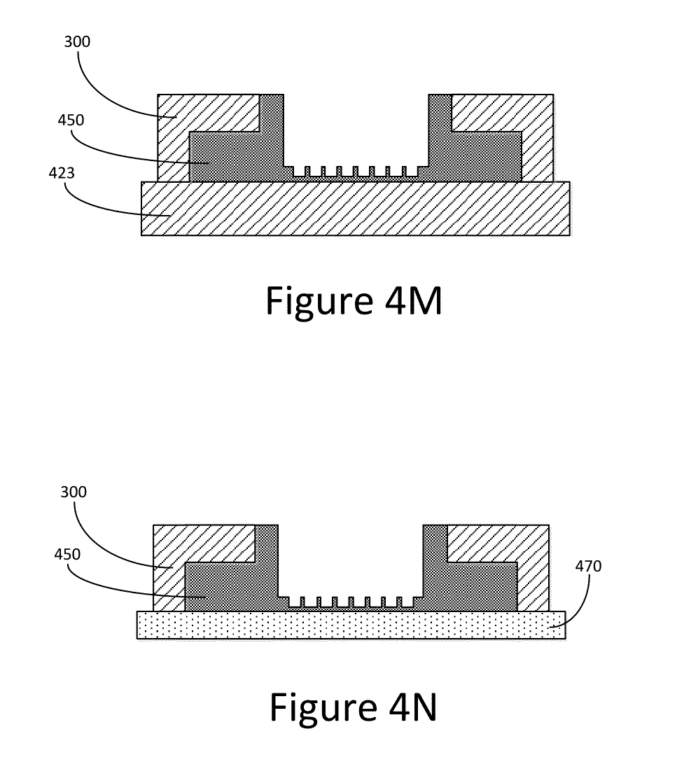

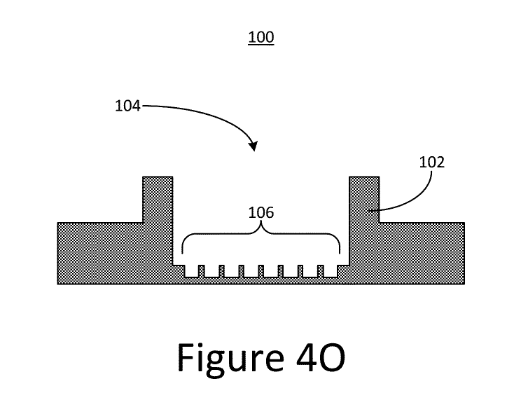

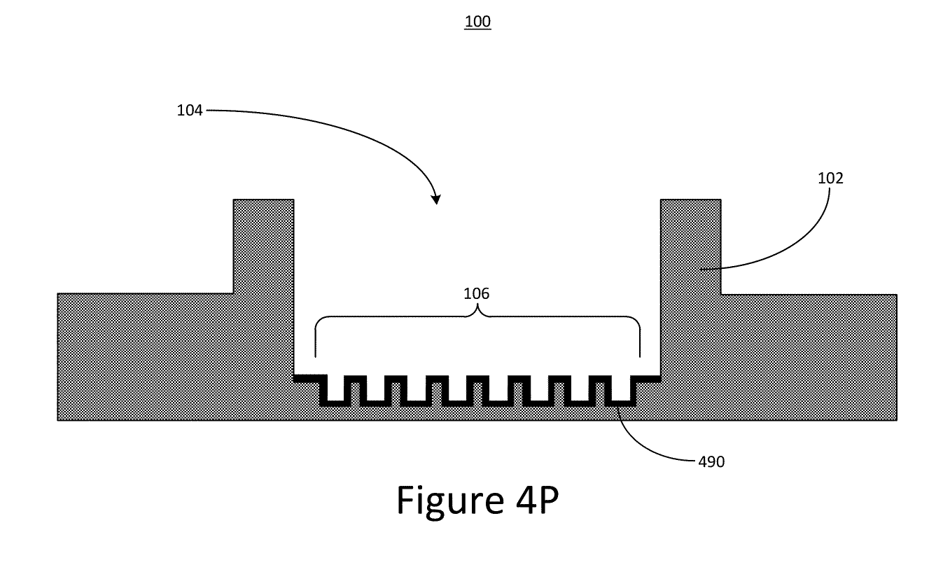

FIGS. 4A-40 show cross-sectional views of example stages of construction of a micro-feature device according to the manufacturing process 200 shown in FIG. 2. FIGS. 4A-40 will be described with reference to the process 200 shown in FIG. 2, the outer mold 300 shown in FIG. 3A, and the inner mold 330 shown in FIG. 3B and FIG. 3C. Referring again to FIG. 2, the process 200 begins with positioning an outer mold in contact with a transparent substrate (stage 202). FIG. 4A shows a first implementation of this stage, and FIG. 4B shows a second implementation of this stage. In particular, FIG. 4A differs from FIG. 4B in that FIG. 4A shows a platform 423 having a high thermal conductivity supporting the substrate 450, while FIG. 4B shows a thermal buffer platform 470 supporting the substrate 450. These differences are discussed further below.

As shown in both FIG. 4A and FIGS. 4B, the outer mold 300 is positioned on top of a substrate 450 such that the substrate 450 is held in place by the outer mold 300. In some implementations, the substrate can fit into the recess 302 (not labeled in FIGS. 4A and 4B) defined in the outer mold. The cutout 304 of the outer mold 300 allows a section of the substrate 450 to remain exposed after the outer mold 300 has been positioned in contact with two opposing side surfaces of the substrate 450, as well as a portion of the upper surface of the substrate 450. As discussed above, in some implementations, the substrate 450 can be formed from an optically clear material that has a coefficient of thermal expansion higher than the coefficient of thermal expansion of the outer mold 300. In some implementations, the substrate 450 can be formed from polystyrene. In some other implementations, the substrate 450 can be formed from polycarbonate, polymethylpentene, cyclic olefin copolymer, or PDMS.

The process 200 includes positioning an inner mold on an upper surface of the transparent substrate (stage 204). FIG. 4C shows the results of this stage for the first implementation of the process 200, while FIG. 4D shows the results of this stage for the second implementation of the process 200. The inner mold 330 is positioned within the cutout 304 of the outer mold 300, such that the macropillars and micropillars of the inner mold 330 are in contact with the substrate 450. For illustrative purposes, only a single macropillar and its associated micropillars are shown in FIGS. 4C and 4D. However, it should be understood that in other implementations, the inner mold 330 can include any number of macropillars and micropillars.

The process 200 includes applying a first platen at a first temperature to the inner mold 330 to form a plurality of macrowells and microwells defined in the transparent substrate 450 (stage 206). FIG. 4E shows the results of this stage for the first implementation of the process 200, while FIG. 4F shows the results of this stage for the second implementation of the process 200. A first platen 460 is aligned with the surface of the inner mold 330 and its temperature is increased. The process 200 also can include applying a second platen 465 to the platform 423. In some implementations, the first platen 460 and the second platen 465 can be components of a commercial molding machine. In the first implementation of the process 200, shown in FIG. 4E, the second platen 465 can be heated and the platform 423 can be formed from a rigid material selected to have a relatively high thermal conductivity, so that heat from the second platen 465 is efficiently transferred to the substrate 450.

In the first implementation of the process 200, shown in FIG. 4E, the first platen 460 can be controlled to between about 400 degrees Fahrenheit and about 510 degrees Fahrenheit. The temperature of the first platen 460 can be selected based on the material of the substrate 450. For example, if the substrate 450 is made from polystyrene, the first platen 460 can be controlled to be about 411 degrees. If the substrate 450 is made from polycarbonate, the first platen 460 can be controlled to be about 492 degrees. If the substrate 450 is made from polymethylpentene, the first platen 460 can be controlled to be about 469 degrees. If the substrate 450 is made from a cyclic olefin polymer such as Zeonor, the first platen 460 can be controlled to be about 479 degrees. If the substrate 450 is made from a cyclic olefin polymer such as Topas, the first platen 460 can be controlled to be about 501 degrees.

In the first implementation of the process 200, shown in FIG. 4E, the second platen 465 can be controlled to between about 300 degrees Fahrenheit and about 410 degrees Fahrenheit. The temperature of the second platen 465 also can be selected based on the material of the substrate 450. For example, if the substrate 450 is made from polystyrene, the second platen 465 can be controlled to be about 310 degrees. If the substrate 450 is made from polycarbonate, the second platen 465 can be controlled to be about 391 degrees. If the substrate 450 is made from polymethylpentene, the second platen 465 can be controlled to be about 368 degrees. If the substrate 450 is made from a cyclic olefin polymer such as Zeonor, the second platen 465 can be controlled to be about 378 degrees. If the substrate 450 is made from a cyclic olefin polymer such as Topas, the second platen 465 can be controlled to be about 400 degrees.

In the second implementation of the process 200, shown in FIG. 4F, the platform 470 is acts as a thermal buffer and the second platen 465 is not heated. In some implementations, the thermal buffer platform 470 can be formed from a polymer material, such as polyether ether ketone (PEEK) or a polyimide, such as Kapton™. The benefits of using the thermal buffer platform 470 and an ambient temperature second platen 465 are described further below in connection with FIG. 4J.

The first platen 460 can be pressed downwards against the substrate 450. In some implementations, the first platen 460 can exert a pressure in the range of about 30 PSI to about 70 PSI on the first substrate. As a result of the pressure and temperature applied to the substrate 460, the inner mold 330 is compressed slightly and the viscosity of the substrate 450 is reduced, allowing the inner mold 330 to pass through the upper surface of the substrate 450. As the material of the substrate 450 is displaced by the inner mold 330, the level of the upper surface of the substrate 450 rises. FIG. 4G shows this effect for the first implementation of the process 200, while FIG. 4H shows this effect for the second implementation of the process 200. The molding process can continue until the inner mold 330 is positioned in contact with the outer mold 300, or until the inner mold 300 reaches a desired depth within the substrate 450. FIG. 4I shows this stage for the first implementation of the process 200, while FIG. 4J shows this stage for the second implementation of the process 200. The void created by the macropillars and micropillars of the inner mold 330 within the transparent substrate 450 defines the macrowells and microwells of the micro-feature device.

In some implementations, air may become trapped at the interface of the inner mold 330 and the substrate 450. Trapped air can result in bubble-like defects in the surface of the finished micro-feature device. In order to prevent air from becoming trapped, the inner mold 330 can be formed from a material that is permeable to gas so that air can pass through the inner mold 330 without becoming trapped in the substrate 450. In some implementations, a gas permeable material such as PDMS can be used to form the inner mold 330.

As discussed above, it can be desirable to fabricate a micro-feature device having a thickness that is substantially uniform across the entire bottom surface to allow microscopes to accurately image cell cultures within the device. Using the thermal buffer platform 470 as shown in FIG. 4J, and maintaining the second platen 465 at ambient temperature rather than applying heat to the second platen 465, can facilitate the fabrication of a device having uniform thickness. For example, the temperature (and, as a result, the viscosity) of the substrate 450 in the vicinity of the micropillars of the inner mold 330 can be actively maintained by the thermal buffer 470. This can produce a more consistent resistance to balance the expansion force of the compressed inner mold 330, which can result in a thin bottom having substantially uniform thickness.

The process 200 includes cooling the outer mold 300 and the inner mold 330 to a second temperature (stage 208). FIG. 4K shows the results of this stage for the first implementation of the process 200, while FIG. 4L shows the results of this stage for the second implementation of the process 200. The first platen 460 and the second platen 465 are removed The inner mold 330, the outer mold 300, and the substrate 450 are then allowed to cool to the ambient temperature under pressure. In some implementations, the pressure applied while the inner mold 330, the outer mold 300, and the substrate 450 are then allowed to cool is the range of about three PSI to about 15 PSI.

The process 200 includes removing the inner mold from the transparent substrate (stage 210). FIG. 4M shows the results of this stage for the first implementation of the process 200, while FIG. 4N shows the results of this stage for the second implementation of the process 200. As discussed above, the inner mold 330 can be formed from a material that has a coefficient of thermal expansion greater than the coefficient of thermal expansion of the substrate 450. In some implementations, this can help to facilitate removal of the inner mold 330 from the substrate 450. For example, during the cooling stage (stage 208), the inner mold 330, the outer mold 300, and the substrate 450 will tend to contract. Selecting the material of the inner mold 330 to have a coefficient of thermal expansion greater than the coefficient of thermal expansion of the substrate 450 allows the inner mold 330 to contract more than the substrate 450. This generates microgaps at the interface of the inner mold 330 and the substrate 450, which allows the inner mold 330 to be more easily removed from the substrate 450 after the cooling process. Removing the inner mold 330 (stage 210) can therefore be accomplished with a lower likelihood of damaging the sidewalls of the microwells formed in the substrate 450. If the inner mold 330 is formed from a material that has a coefficient of thermal expansion less than or equal to the coefficient of thermal expansion of the substrate 450, it may be necessary to add a release agent to the inner surfaces of the substrate 450 in order to safely remove the inner mold 330. However, such agents can interfere with the culture of cells in the resulting device, and therefore may be undesirable.

The process 200 includes removing the transparent substrate from the outer mold (stage 212). The resulting micro-feature device 100 is shown in FIG. 4O. As discussed above, the outer mold 300 can be formed from a material that has a coefficient of thermal expansion less than the coefficient of thermal expansion of the substrate 450. In some implementations, this can help to facilitate removal of the substrate 450 from the outer mold 300 by generating microgaps at the interface of the substrate 450 and the outer mold 300. The risk of damaging the transparent substrate 450 during the removal process is therefore decreased.

In some implementations, the process 200 also can include applying a coating to the microwells 106 of the device 100. The material used to coat the microwells 106 can be selected, for example, to be compatible with the cells that are to be cultured in the microwells 106. In some implementations, the coating can be a polymer material, such as silicone, polyuerathane, or epoxy. In some implementations, the polymer material can be PDMS. FIG. 4P shows the micro-feature device 100 after a coating 490 has been applied to the microwells 106.

As shown in FIG. 4P, the coating 490 covers the bottom and sidewalls of the microwells 106. In some implementations, it may not be necessary to cover the sidewalls of the macrowells 104 with the coating, because cell culture is carried out primarily in within the microwells 106. In some implementations, the sidewalls of the microwells 106 also may not be coated. The coating 490 can be applied conformally so that the coating 490 does not completely fill the microwells 106. In some implementations, the coating 490 can have a thickness in the range of about 0.5 microns to about 25 microns.

In some implementations, the material forming the coating 490 can be applied a liquid. For example, the coating 490 can be applied by spray coating a liquid material, such as liquid PDMS. In some implementations, a solvent or other thinning agent may be added to the material that will form the coating 490. For example, the solvent can thin the material, thereby facilitating the formation of a thin conformal coating 490. In some implementations, the coating 490 can be applied by ink jet printing a layer of material over the microwells 106. In still other implementations, a different precision deposition process may be used. The coating 490 can then be cured to solidify the material forming the coating 490. In some implementations, the microwells 106 may be preheated prior to the deposition of the coating 490 such that at least a portion of the coating 490 begins to cure upon contact with the microwells 106. In some other implementations employing other coatings, the coating 490 may be cured by other means, such as the application of UV light.

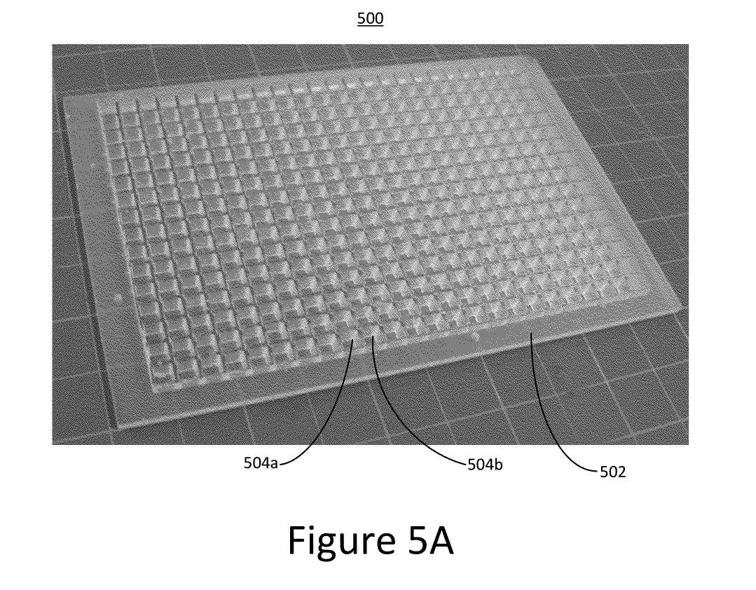

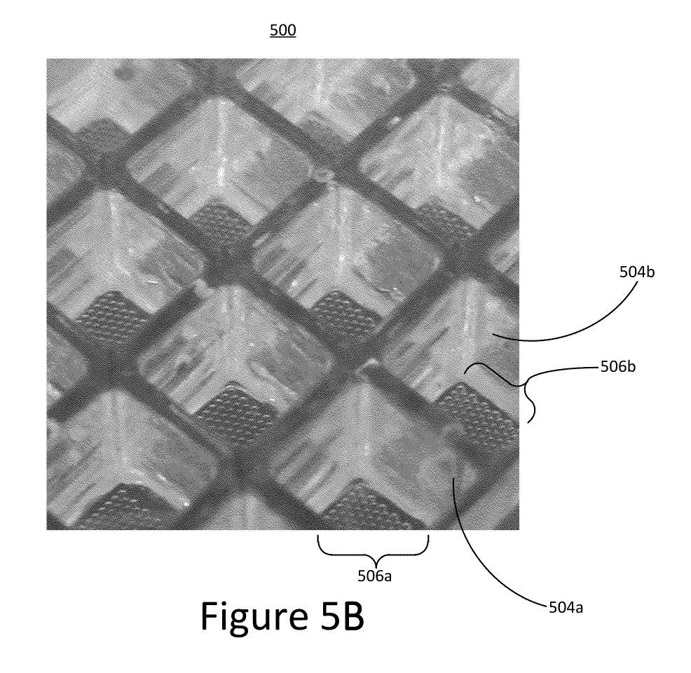

FIG. 5A shows a photograph of a micro-feature device 500 that can be fabricated according to the manufacturing process shown in FIG. 2, according to an illustrative implementation. The micro-feature device 500 is similar to the micro-feature device 100 shown in FIG. 1A. For example, the micro-feature device 500 includes a substrate 502 in which macrowells, such as the macrowells 504a and 504b (generally referred to as macrowells 504), are defined. Although not visible in FIG. 5A, each macrowell 504 also can include a plurality of microwells defined in the bottom surface of the macrowell 504.

FIG. 5B shows an enlarged view of a portion of the micro-feature device 500 shown in FIG. 5A, according to an illustrative implementation. The macrowells 504 are shown under magnification. Each macrowell 504 includes a respective plurality of microwells 506a and 506b (generally referred to as microwells 506). The microwells 506 are defined in the bottom surface of each macrowell 504.

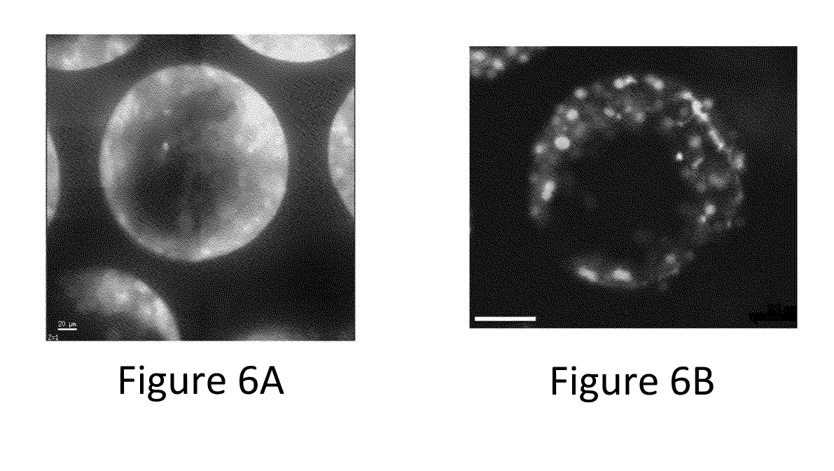

FIGS. 6A and 6B show images of cell cultures within a microwell of a micro-feature device, according to an illustrative implementation. To produce these images, devices such as the micro-feature device 100 shown in FIG. 1 were sterilized by overnight ethylene oxide treatment and wetted by addition of 30 μL sterile water to each well. Air bubbles were removed by three rounds of vacuum application for 10 min and mild tapping against a solid surface followed by overnight incubation at 37 C. After confirmation of wetting by microscopy, macrowells were collagen coated by addition of 4 μg/mL rat tail collagen I in 0.02 N acetic acid for 12 hours at 37 C. Macrowells were then washed thrice with Plating media (Bioreclamation IVT) and seeded with 30,000 primary human hepatocytes in a 2,000 cells/μL solution of media following thawing by manufacturer's instructions (Bioreclamation IVT). Following seeding, media volume was adjusted to 50 μL per well with 25 μL refreshed every 48-72 hours. At 11 days post seed, macrowells were washed with media and stained with 2 μg/mL 5-(and-6)-Carboxy-2′,7′-Dichlorofluorescein Diacetate (DCFDA, Life Technologies) and 10 μg/mL Hoechst 33342 for 10 min. After washing with Plate media, inter-hepatocyte bile canaliculi filled with fluorescent DCFDA were imaged with a 40× objective on a Deltavision Elite (GE Healthcare). DCFDA shows as green, nuclei as blue; scale bar represents 50 μM. After 21 days, macrowells were washed with serum free Maintenance media (Bioreclamation IVT) and stained with 2 μM Calcein AM, 4 μM Ethidium Homodimer (Live/Dead kit, Life Technologies) and 10 μg/mL Hoechst 33342 (Sigma) in Maintenance media for 30 min at 37 C and then washed thrice with Plating media. Live, stained macrowells were imaged with a 40× objective on a Deltavision Elite, using the panels function to collect and stitch together four Fields-of-View to capture all hepatocytes within one microwell. Live cells stain green, dead cells stain red, and nuclei stain blue; scale bar represents 20 μm.

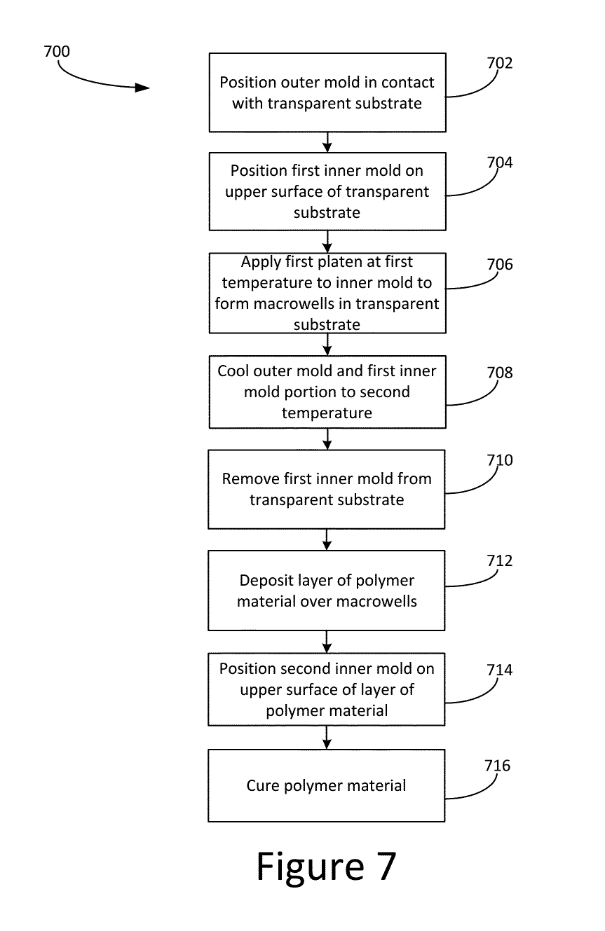

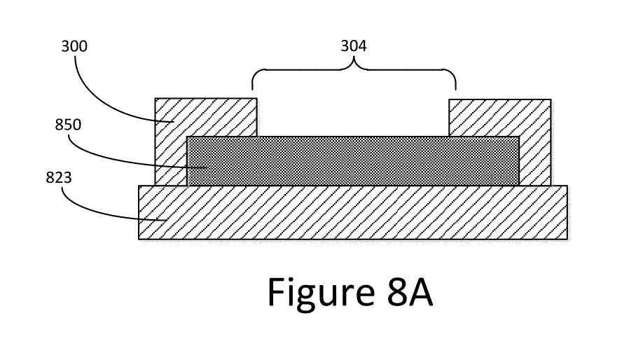

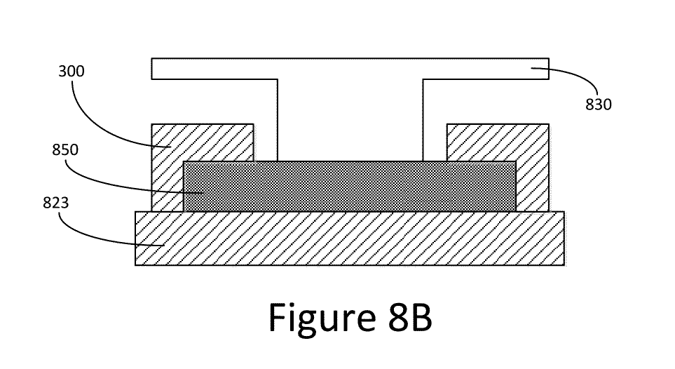

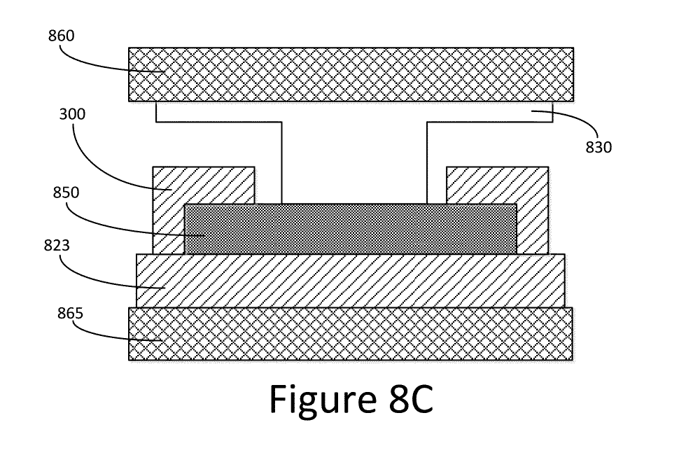

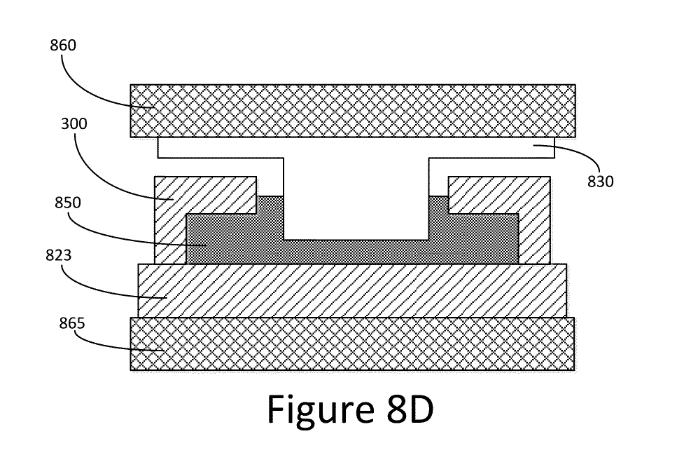

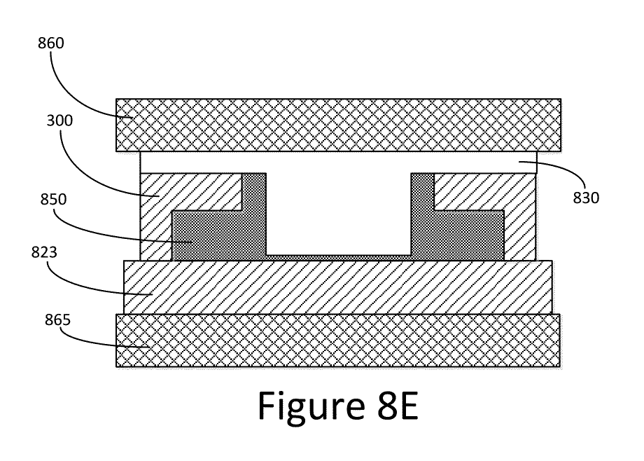

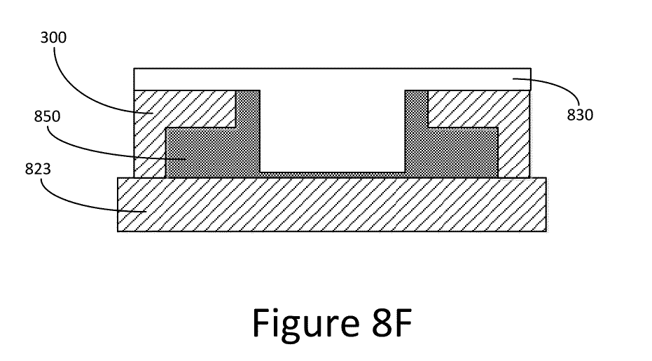

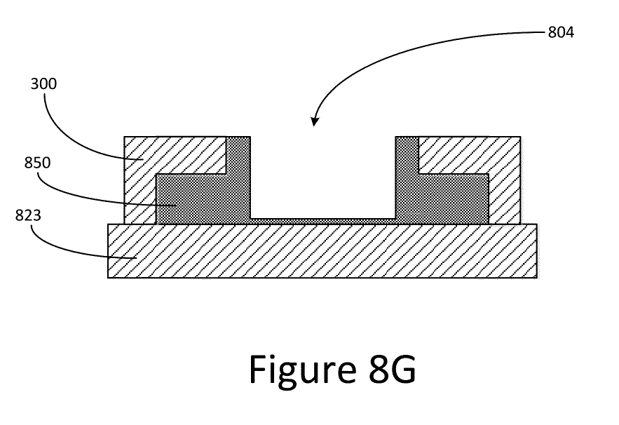

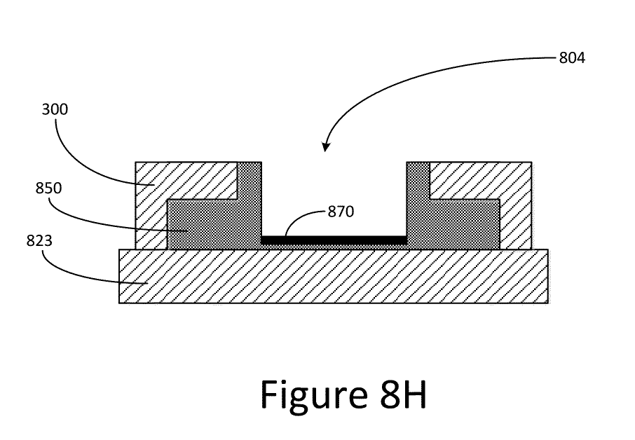

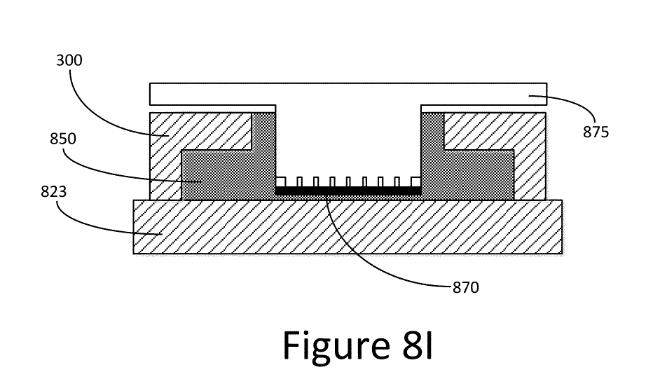

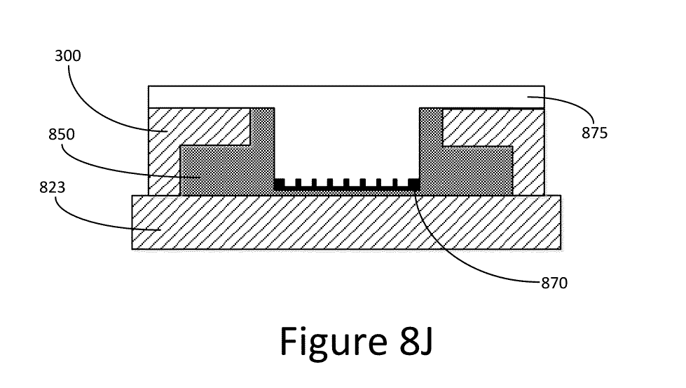

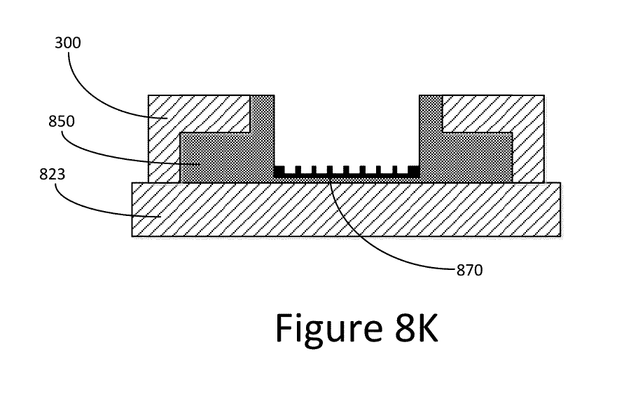

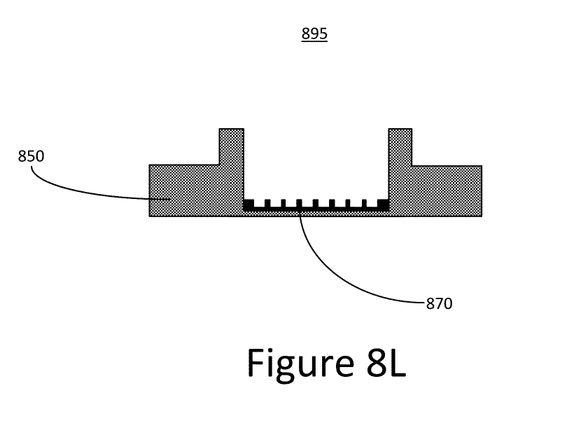

FIG. 7 shows a flow chart for a process 700 of manufacturing a micro-feature device, according to an illustrative implementation. FIGS. 8A-8L show cross-sectional views of example stages of construction of a micro-feature device according to the manufacturing process shown in FIG. 7. FIGS. 7 and 8A-8L are discussed together below. The process 700 is similar to the process 200 discussed above in connection with FIG. 2. However, the process 700 is distinguished from the process 200 in that the process 700 can be used to fabricate a micro-feature device in which the microwells are formed using replica molding in a material such as PDMS, while the macrowells are formed using hot embossing in a different material, such as polystyrene. In brief overview, the process 700 includes positioning an outer mold in contact with a transparent substrate (stage 702). The process 700 includes positioning a first inner mold on an upper surface of the transparent substrate (stage 704). The process 700 includes applying a first platen at a first temperature to the first inner mold to form a plurality of macrowells defined in the transparent substrate (stage 706). The process 700 includes cooling the outer mold and the first inner mold to a second temperature (stage 708). The process 700 includes removing the first inner mold from the transparent substrate (stage 710). The process 700 includes depositing a layer of polymer material onto a bottom surface of each macrowell (stage 712). The process 700 includes positioning a second inner mold on an upper surface of the layer of polymer material to cause the second inner mold to form a respective plurality of microwells defined in a respective lower surface of each macrowell (stage 714). The process 700 includes curing the layer of polymer material (stage 716).

Referring again to FIG. 7, the process 700 begins with positioning an outer mold in contact with a transparent substrate (stage 702). FIG. 8A shows the results of this stage. In some implementations, the outer mold 300 described above in connection with FIG. 3A can be used in the process 700. The outer mold 300 is positioned on top of a substrate 850 such that the substrate 850 is held in place by the outer mold 300. A platform 823 is positioned beneath the substrate 850. In some implementations, the substrate 850 can fit into the recess 302 (not labeled in FIG. 8A) defined in the outer mold 300. The cutout 304 of the outer mold 300 allows a section of the substrate 850 to remain exposed after the outer mold 300 has been positioned in contact with two opposing side surfaces of the substrate 850, as well as a portion of the upper surface of the substrate 850. As discussed above, in some implementations, the substrate 850 can be formed from an optically clear material that has a coefficient of thermal expansion higher than the coefficient of thermal expansion of the outer mold 300. In some implementations, the substrate 850 can be formed from polystyrene. In some other implementations, the substrate 850 can be formed from polycarbonate, polymethylpentene, cyclic olefin copolymer, or PDMS.

The process 700 includes positioning a first inner mold on an upper surface of the transparent substrate (stage 704). FIG. 8B shows the results of this stage. The first inner mold 830 is positioned within the cutout 304 of the outer mold 300. In some implementations, the first inner mold can be similar to the inner mold 330 described above in connection with FIG. 3B, except that the first inner mold 830 does not include micropillars on each macropillar. Because the inner mold 830 includes only macropillars, the macropillars of the first inner mold 830 are in contact with the substrate 850. For illustrative purposes, only a single macropillar is shown in FIGS. 8B. However, it should be understood that in other implementations, the first inner mold 830 can include any number of macropillars.

The process 700 includes applying a first platen at a first temperature to the first inner mold 830 to form a plurality of macrowells defined in the transparent substrate 850 (stage 706). FIG. 8C shows the results of this stage. A first platen 860 is aligned with the surface of the first inner mold 830 and its temperature is increased. The process 700 also can include applying a second platen 865 to the platform 823. In some implementations, the first platen 860 and the second platen 865 can be components of a commercial molding machine. The first platen 860 and the second platen 865 can be heated, and the platform 823 can be formed from a rigid material selected to have a relatively high thermal conductivity, so that heat from the second platen 865 is efficiently transferred to the substrate 850.

In some implementations, the first platen 860 can be controlled to between about 400 degrees Fahrenheit and about 510 degrees Fahrenheit. The temperature of the first platen 860 can be selected based on the material of the substrate 850. For example, if the substrate 850 is made from polystyrene, the first platen 860 can be controlled to be about 411 degrees. If the substrate 850 is made from polycarbonate, the first platen 860 can be controlled to be about 492 degrees. If the substrate 850 is made from polymethylpentene, the first platen 460 can be controlled to be about 469 degrees. If the substrate 850 is made from a cyclic olefin polymer such as Zeonor, the first platen 860 can be controlled to be about 479 degrees. If the substrate 850 is made from a cyclic olefin polymer such as Topas, the first platen 860 can be controlled to be about 501 degrees.

The second platen 865 can be controlled to between about 300 degrees Fahrenheit and about 410 degrees Fahrenheit. The temperature of the second platen 865 also can be selected based on the material of the substrate 850. For example, if the substrate 850 is made from polystyrene, the second platen 865 can be controlled to be about 310 degrees. If the substrate 850 is made from polycarbonate, the second platen 865 can be controlled to be about 391 degrees. If the substrate 850 is made from polymethylpentene, the second platen 865 can be controlled to be about 368 degrees. If the substrate 850 is made from a cyclic olefin polymer such as Zeonor, the second platen 865 can be controlled to be about 378 degrees. If the substrate 850 is made from a cyclic olefin polymer such as Topas, the second platen 865 can be controlled to be about 400 degrees.

The first platen 860 can be pressed downwards against the substrate 850. In some implementations, the first platen 860 can exert a pressure in the range of about 30 PSI to about 70 PSI on the first substrate. As a result of the pressure and temperature applied to the substrate 860, the inner mold 830 is compressed slightly and the viscosity of the substrate 850 is reduced, allowing the inner mold 830 to pass through the upper surface of the substrate 850. As the material of the substrate 850 is displaced by the inner mold 830, the level of the upper surface of the substrate 850 rises. FIG. 8D shows this effect. The molding process can continue until the inner mold 830 reaches a desired depth within the substrate 850. FIG. 8E shows the first inner mold 830 in its fully depressed position. The void created by the macropillars of the inner mold 830 within the transparent substrate 850 defines the macrowells of the micro-feature device.

In some implementations, air may become trapped at the interface of the first inner mold 830 and the substrate 850. Trapped air can result in bubble-like defects in the surface of the finished micro-feature device. In order to prevent air from becoming trapped, the inner mold 830 can be formed from a material that is permeable to gas so that air can pass through the inner mold 830 without becoming trapped in the substrate 850. In some implementations, a gas permeable material such as PDMS can be used to form the inner mold 830.

The process 700 includes cooling the outer mold 300 and the inner mold 330 to a second temperature (stage 708). FIG. 8F shows the results of this stage. The first platen 860 and the second platen 865 are removed. The inner mold 830, the outer mold 300, and the substrate 850 are then allowed to cool to the ambient temperature under pressure. In some implementations, the pressure applied while the inner mold 830, the outer mold 300, and the substrate 850 are allowed to cool is the range of about three PSI to about 15 PSI.

The process 700 includes removing the first inner mold from the transparent substrate (stage 710). FIG. 8G shows the results of this stage. As discussed above, the first inner mold 830 can be formed from a material that has a coefficient of thermal expansion greater than the coefficient of thermal expansion of the substrate 850. In some implementations, this can help to facilitate removal of the first inner mold 830 from the substrate 850. For example, during the cooling stage (stage 708), the first inner mold 830, the outer mold 300, and the substrate 850 will tend to contract. Selecting the material of the first inner mold 830 to have a coefficient of thermal expansion greater than the coefficient of thermal expansion of the substrate 850 allows the first inner mold 830 to contract more than the substrate 850. This generates microgaps at the interface of the first inner mold 830 and the substrate 850, which allows the first inner mold 830 to be more easily removed from the substrate 850 after the cooling process. FIG. 8G shows a macrowell 804 formed after removal of the first inner mold 830.

The process 700 includes depositing a layer of polymer material onto a bottom surface of each macrowell (stage 712). FIG. 8H shows the results of this stage. The bottom surface of the macrowell 804 is covered with a layer of polymer material 870. In some implementations, the layer of polymer material can include at least one of silicone, polyurethane, and epoxy. In some implementations, the layer of polymer material can include PDMS. In some implementations, the layer of polymer material 870 can be deposited as a liquid. A liquid layer of polymer material 870 may be more easily molded than a solid layer of polymer material, and therefore molding of the layer of polymer material 870 can be carried out without the use of heated platens. However, in some implementations, heated platens or another heat source may be used to facilitate cross-linking of the polymer material in order to allow the polymer material to retain its molded shape.

The process 700 includes positioning a second inner mold on an upper surface of the layer of polymer material to cause the second inner mold to form a respective plurality of microwells defined in a respective lower surface of each macrowell (stage 714). FIG. 8I shows the second inner mold 875 positioned on the layer of polymer material 870. As shown, the second inner mold 875 includes micropillars similar to those described above in connection with the inner mold 330 shown in FIG. 3B. Each of the micropillars corresponds to a microwell to be formed in the micro-feature device.

In some implementations, the second inner mold 875 can also be formed from a polymer material, such as PDMS. In implementations in which both the second inner mold 875 and the deposited layer of polymer material 870 are formed from PDMS, the second inner mold 875 can be treated to prevent adhesion of the layer of polymer material 870 to the PDMS forming the second inner mold 875. For example, the surface of the second inner mold 875 can be fluorinated to prevent adhesion. In other implementations, the second inner mold 875 can be formed from a different material, such as polyurethane, epoxy, or other elastomers. To prevent the formation of air bubbles in the layer of polymer material 870, the second inner mold 875 also can be formed from a material that is permeable to gas, as discussed in greater detail above. The second inner mold 875 can be pressed into the layer of polymer material 870. As the layer of polymer material 870 is displaced by the second inner mold 875, the level of the upper surface of the layer of polymer material 870 rises. The voids created by the micropillars of the second inner mold 875 within the layer of polymer material 870 define the microwells of the micro-feature device, as shown in FIG. 8J. In some implementations, the second inner mold 875 can be pressed into the layer of polymer material 870 such that the thickness of the layer underneath each microwell in the layer of polymer material 870 is in the range of about 0.5 microns to about 25 microns.

The process 700 includes curing the layer of polymer material (stage 716). In some implementations, the layer of polymer material 870 can be cured while the second inner mold 875 is still pressed into the layer of polymer material 870, so that the layer of polymer material 870 can retain its shape during the curing process. The curing process can solidify the layer of polymer material 870 and bond the layer of polymer material 870 to the underlying substrate 850. In some implementations, the layer of polymer material 1870 can be cured by exposing the layer of polymer material to heat and pressure. In other implementations, the layer of polymer material 870 can be cured in other ways. For example, the layer of polymer material 870 can be treated with a photosensitive material and exposed to ultraviolet light to cure the layer of polymer material 870.

The process 700 can include removing the platform 823, the outer mold 300, and the second inner mold 875. The resulting micro-feature device 895 is shown in FIG. 8L. As discussed above, the outer mold 300 can be formed from a material that has a coefficient of thermal expansion less than the coefficient of thermal expansion of the substrate 850. In some implementations, this can help to facilitate removal of the substrate 850 from the outer mold 300 by generating microgaps at the interface of the substrate 850 and the outer mold 300. The risk of damaging the transparent substrate 850 during the removal process is therefore decreased. Similarly, the second inner mold 875 can be formed from a material that has a coefficient of thermal expansion greater than the coefficient of thermal expansion of the layer of polymer material 870, thereby facilitating removal of the second inner mold 875 and reducing the likelihood of damaging the layer of polymer material 1870.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. In certain circumstances, multitasking and parallel processing may be advantageous. Moreover, the separation of various system components in the implementations described above should not be understood as requiring such separation in all implementations. Additionally, other implementations are within the scope of the following claims. In some cases, the actions recited in the claims can be performed in a different order and still achieve desirable results.

What is claimed is:

1. A micro-feature device comprising:
a substantially rigid transparent substrate;
a plurality of macrowells defined in the transparent substrate, wherein each macrowell has a width in the range of about one millimeter to about 35 millimeters and a depth in the range of about two millimeters to about 12 millimeters, and wherein each macrowell includes a respective plurality of microwells defined in a respective lower surface of the macrowell, each microwell having a width in the range of about 50 microns to about 500 microns and a depth in the range of about 50 microns to about 500 microns.

2. The micro-feature device of claim 1, wherein each pair of adjacent macrowells is separated by a distance in the range of about 0.5 millimeters to about five millimeters.

3. The micro-feature device of claim 1, wherein each macrowell has a height-to-width ratio in the range of about 1:1 to about 5:1.

4. The micro-feature device of claim 1, wherein each pair of adjacent microwells is separated by a distance in the range of about 50 microns to about 200 microns.

5. The micro-feature device of claim 1, wherein each microwell has a height-to-width ratio in the range of about 1:1 to about 5:1.

6. The micro-feature device of claim 1, wherein the transparent substrate comprises at least one of polystyrene, polycarbonate, polymethylpentene, a cyclic olefin copolymer, and polydimethylsiloxane (PDMS).

7. The micro-feature device of claim 1, wherein a distance between a lower surface of the transparent substrate and a lower surface of at least one microwell of the plurality of microwells is between about 25 microns and about 200 microns.

8. The micro-feature device of claim 7, wherein a distance between a lower surface of the transparent substrate and a lower surface of each microwell of the plurality of microwells is substantially uniform.

9. The micro-feature device of claim 1, wherein a bottom surface of each microwell comprises a layer of polymer material.

10. The micro-feature device of claim 9, wherein the layer of polymer material at the bottom of each microwell has a thickness in the range of about 0.5 microns to about 25 microns.

11. The micro-feature device of claim 9, wherein the layer of polymer material comprises at least one of polyurethane and epoxy.

12. The micro-feature device of claim 9, wherein the layer of polymer material comprises silicone.

13. The micro-feature device of claim 12, wherein the layer of polymer material comprises PDMS.

14. A method of fabricating a micro-feature device, comprising:
positioning an outer mold in contact with at least two opposing side surfaces and a portion of an upper surface of a substantially rigid transparent substrate, wherein a coefficient of thermal expansion of the outer mold is less than a coefficient of thermal expansion of the transparent substrate;
positioning an inner mold on the upper surface of the transparent substrate, wherein the coefficient of thermal expansion of the transparent substrate is less than a coefficient of thermal expansion of the inner mold;
applying a first platen at a first temperature to the inner mold to cause the inner mold to form a plurality of macrowells defined in the transparent substrate, each macrowell having a width in the range of about one millimeter to about 35 millimeters, a depth in the range of about two millimeters to about 12 millimeters, and a respective plurality of microwells defined in a respective lower surface of the macrowell, each microwell having a width in the range of about 50 microns to about 500 microns and a depth in the range of about 50 microns to about 500 microns;
cooling the outer mold, the transparent substrate, and the inner mold to a second temperature, lower than the first temperature;
removing the inner mold from the transparent substrate; and
removing the transparent substrate from the outer mold.

15. The method of claim 14, further comprising selecting a first material for the inner mold, wherein:
the first material is selected to be softer than the transparent substrate; and
the first material has a durometer measurement in the range of about 30A to about 80A.

16. The method of claim 14, further comprising selecting a second material for the outer mold, wherein:
the second material is selected to be harder than the transparent substrate; and
the second material has a Mohs hardness in the range of about 2 to about 3 or a Rockwell hardness in the range of about R120 to about R130.

17. The method of claim 14, wherein the inner mold is formed from polydimethylsiloxane (PDMS).

18. The method of claim 14, wherein the inner mold includes a plurality of macropillars extending outward from a surface of the inner mold, each macropillar further including a respective plurality of micropillars extending outwards from a respective surface of the macropillar.

19. The method of claim 18, wherein each macropillar has a width in the range of about one millimeter to about 35 millimeters and a height in the range of about two millimeters to about 12 millimeters.

20. The method of claim 18, wherein each pair of adjacent macropillars is separated by a distance in the range of about 0.5 millimeters to about five millimeters.

21. The method of claim 18, wherein each macropillar has a height-to-width ratio in the range of about 1:1 to about 5:1.

22. The method of claim 18, wherein each micropillar has a width in the range of about 50 microns to about 500 microns and a height in the range of about 50 microns to about 500 microns.

23. The method of claim 18, wherein each pair of adjacent micropillars is separated by a distance in the range of about 25 microns to about 200 microns.

24. The method of claim 18, wherein each micropillar has a height-to-width ratio in the range of about 1:1 to about 5:1.

25. The method of claim 14, wherein the inner mold comprises a material that is permeable to gas.

26. The method of claim 14, wherein the transparent substrate is formed from at least one of polystyrene, polycarbonate, polymethylpentene, and cyclic olefin copolymer.

27. The method of claim 14, further comprising:
providing a supporting platform in contact with a lower surface of the transparent substrate; and
applying a second platen at a second temperature to the supporting platform.

28. The method of claim 14, further comprising:
providing a supporting platform comprising a thermal buffer layer in contact with a lower surface of the transparent substrate; and
applying a second platen at an ambient temperature to the supporting platform.

29. The method of claim 28, wherein the thermal buffer layer is formed from one of polyether ether ketone (PEEK) or a polyimide film.

30. The method of claim 14, further comprising depositing a coating comprising a polymer material on a bottom surface of each microwell.

31. The method of claim 30, wherein the coating comprises at least one of polyurethane and epoxy.

32. The method of claim 30, wherein the coating comprises silicone.

33. The method of claim 31, wherein the coating comprises PDMS.

34. The method of claim 30, wherein the coating further comprises a solvent.

35. The method of claim 30, wherein the coating is a conformal coating applied over the bottom surface and sidewalls of each microwell.

36. The method of claim 30, wherein the coating has a thickness in the range of about 0.5 microns to about 25 microns.

37. A method of fabricating a micro-feature device, comprising:
positioning an outer mold in contact with at least two opposing side surfaces and a portion of an upper surface of a substantially rigid transparent substrate, wherein a coefficient of thermal expansion of the outer mold is less than a coefficient of thermal expansion of the transparent substrate;
positioning a first inner mold on the upper surface of the transparent substrate, wherein the coefficient of thermal expansion of the transparent substrate is less than a coefficient of thermal expansion of the first inner mold;
applying a first platen at a first temperature to the first inner mold to cause the first inner mold to form a plurality of macrowells defined in the transparent substrate, each macrowell having a width in the range of about one millimeter to about 35 millimeters and a depth in the range of about two millimeters to about 12 millimeters;
cooling the outer mold, the transparent substrate, and the first inner mold to a second temperature, lower than the first temperature;
removing the first inner mold from the transparent substrate;
depositing a layer of polymer material onto a bottom surface of each macrowell;
positioning a second inner mold on an upper surface of the layer of polymer material to cause the second inner mold to form a respective plurality of microwells defined in a respective lower surface of each macrowell, each microwell having a width in the range of about 50 microns to about 500 microns and a depth in the range of about 50 microns to about 500 microns; and
curing the layer of polymer material.

38. The method of claim 37, wherein the layer of polymer material at the bottom of each microwell has a thickness in the range of about 0.5 microns to about 25 microns.

39. The method of claim 37, wherein the second inner mold comprises at least one of polyurethane and epoxy.

40. The method of claim 37, wherein the second inner mold comprises an elastomer.

41. The method of claim 40, wherein the second inner mold comprises at least one of PDMS and fluorinated PDMS.

42. The method of claim 37, wherein the second inner mold comprises a material that is permeable to gas.

43. The method of claim 37, wherein the layer of polymer material comprises at least one of polyurethane and epoxy.

44. The method of claim 37, wherein the layer of polymer material comprises silicone.

45. The method of claim 44, wherein the layer of polymer material comprises PDMS.
